(19) United States
(12) Patent Application Publication
(10) Pub. No.: US 2018/0179189 A1
(43) Pub. Date: Jun. 28, 2018

(54) ADIPATE FORMS AND COMPOSITIONS OF BIARYL INHIBITORS OF BRUTON'S TYROSINE KINASE

(71) Applicant: BIOGEN MA INC., Cambridge, MA (US)

(72) Inventors: J. Michael MacPhee, Cambridge, MA (US); Robbie Chen, Cambridge, MA (US); Steven Ferguson, Cambridge, MA (US); Lloyd Franklin, Cambridge, MA (US); Tamera L. Mack, Cambridge, MA (US)

(21) Appl. No.: 15/580,561

(22) PCT Filed: Jun. 10, 2016
(86) PCT No.: PCT/US2016/036952

§ 371 Date: Dec. 7, 2017

Related U.S. Application Data
(60) Provisional application No. 62/173,896, filed on Jun. 10, 2015.

(51) Int. Cl. C07D 403/14

(52) U.S. Cl. CPC C07D 403/14

(57) ABSTRACT

The present invention provides compounds and compositions thereof which are useful as inhibitors of Bruton's tyrosine kinase and which exhibit desirable characteristics for the same.

### CROSS REFERENCE TO RELATED APPLICATIONS

The present application claims priority to U.S. provisional patent application No. 62/173,896, filed Jun. 10, 2015, the entire contents of which are hereby incorporated by reference.

### BACKGROUND OF THE INVENTION

Protein kinases are a large multigene family consisting of more than 500 proteins which play a critical role in the development and treatment of a number of human diseases in oncology, neurology and immunology. The Tec kinases are non-receptor tyrosine kinases which consists of five members (Tec (tyrosine kinase expressed in hepatocellular carcinoma), Btk (Bruton's tyrosine kinase), Itk (interleukin-2 (IL-2)-inducible T-cell kinase; also known as Emt or Tsk), Rlk (resting lymphocyte kinase; also known as Txk) and Bmx (bone-marrow tyrosine kinase gene on chromosome X; also known as Etk)) and are primarily expressed in haematopoietic cells, although expression of Bmx and Tec has been detected in endothelial and liver cells. Tec kinases (Itk, Rlk and Tec) are expressed in T cell and are all activated downstream of the T-cell receptor (TCR). Btk is a downstream mediator of B cell receptor (BCR) signaling which is involved in regulating B cell activation, proliferation, and differentiation. More specifically, Btk contains a PH domain that binds phosphatidylinositol (3,4,5)-trisphosphate (PIP3). PIP3 binding induces Btk to phosphorylate phospholipase C (PLCγ), which in turn hydrolyzes PIP2 to produce two secondary messengers, inositol triphosphate (IP3) and diacylglycerol (DAG), which activate protein kinase PKC, which then induces additional B-cell signaling. Mutations that disable Btk enzymatic activity result in XLA syndrome (X-linked agammaglobulinemia), a primary immunodeficiency. Given the critical roles which Tec kinases play in both B-cell and T-cell signaling, Tec kinases are targets of interest for autoimmune disorders.

Consequently, there is a great need in the art for effective inhibitors of Btk. The present invention fulfills these and other needs.

### SUMMARY OF THE INVENTION

It has now been found that novel forms of the present invention, and compositions thereof, are useful as inhibitors of one or more protein kinases and exhibit desirable characteristics for the same. In general, acid addition forms or freebase forms, and pharmaceutically acceptable compositions thereof, are useful for treating or lessening the severity of a variety of diseases or disorders as described in detail herein.

### BRIEF DESCRIPTION OF THE DRAWINGS

FIG. 1 shows the XRPD patterns of Compound 2 Type I and Compound 2 Type II along with adipic acid and Compound 1 Type A.

FIG. 2 shows DSC/TGA data of Compound 2 Type I.

FIG. 3 shows DSC/TGA data of Compound 2 Type II.

FIG. 4 shows TGA/DSC data for Compound 2 Type II.

FIG. 5 provides the XRPD pattern for the scale-up sample of Compound 2 Type I obtained by the procedure of Example 4.

FIG. 6 provides TGA and DSC data for Compound 2 Type I obtained by the procedure of Example 4.

FIG. 7 provides the DVS result showing a water uptake of 0.3% at 25° C./80% RH, indicating Compound 2 Type I is slightly hygroscopic.

FIG. 8 shows the XRPD pattern of Compound 2 Type I prepared using the procedure of Example 5.

FIG. 9 shows TGA and DSC data Compound 2 Type I prepared using the procedure of Example 5.

FIG. 10 shows the XRPD pattern for the Compound 2 Type II obtained by the procedure of Example 6.

FIG. 11 shows the TGA and DSC data for Compound 2 Type II obtained by the procedure of Example 6.

FIG. 12 provides the DVS result, which showed a water uptake of 0.3% at 25° C./80% RH, indicating that Compound 2 Type II is slightly hygroscopic.

FIG. 13 provides solubility data for Compound 2 Type II and Compound 2 Type I.

FIG. 14 shows the three-dimensional structure of Compound 2 Type I single crystal.

FIG. 15 shows the unit cell of Compound 2 Type I single crystal.

FIG. 16 shows the three-dimensional structure of Compound 2 Type II single crystal.

FIG. 17 shows the unit cell of Compound 2 Type II single crystal.

### DETAILED DESCRIPTION OF THE INVENTION

### General Description of Certain Aspects of the Invention:

PCT patent publication WO2015/089337 (PCT application PCT/US14/69853, filed Dec. 11, 2014 (“the '853 application”)), the entirety of which is hereby incorporated herein by reference, describes certain Btk inhibitor compounds. Such compounds include 3-isopropoxy-N-(2-methyl-4-(2-((1-methyl-1H-pyrazol-4-yl)amino)pyrimidin-4-yl)benzyl)azetidine-1-carboxamide:

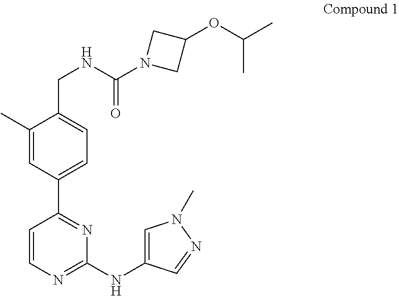 [SMILES: Cc1cc(-c2ccnc(Nc3cnn(C)c3)n2)ccc1CNC(=O)N1CC(OC(C)C)C1]

Compound 1, which is a freebase, is designated as compound number I-21 in the '853 application. The synthesis of compound 1 is described in detail at Example 21 of the '853 application, which is reproduced herein for ease of reference.

Compound 1 has shown potency against BTK in in vitro and in vivo assays of BTK inhibition (see, e.g., Tables 1 and 2 of the '853 application). For example, the '853 application reports that Compound 1 has an IC₅₀<10 nM as measured in an in vitro Btk kinase assay and an IC₅₀<500 nM as measured in a pBTK assay. Accordingly, compound 1 is useful for treating one or more disorders associated with activity of BTK.

It would be desirable to provide an acid addition product or solid form of compound 1 that imparts characteristics such as improved aqueous solubility, stability, absorption, bioavailability, and ease of formulation. Accordingly, the present invention provides both free base forms and acid addition forms of compound 1.

### 1. Compound 2 (Adipic Acid×Compound 1)

According to one embodiment, the present invention provides a chemical species Compound 2 comprising Compound 1 and adipic acid.

In some embodiments, Compound 2 is depicted as:

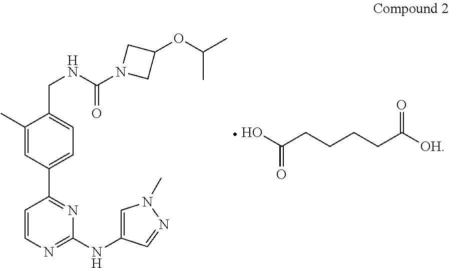 [SMILES: Cc1cc(-c2ccnc(Nc3cnn(C)c3)n2)ccc1CNC(=O)N1CC(OC(C)C)C1 ; O=C(O)CCCCC(=O)O]

It is contemplated that Compound 2 can exist in a variety of solid forms. When Compound 2 is in solid form, said compound may be amorphous, crystalline, or a mixture thereof. Exemplary solid forms are described in more detail below.

In some embodiments, the present invention provides Compound 2 substantially free of impurities. As used herein, the term “substantially free of impurities” means that the compound contains no significant amount of extraneous matter. Such extraneous matter may include excess adipic acid, excess compound 1, residual solvents, or any other impurities that may result from the preparation of, and/or isolation of, Compound 2. In certain embodiments, at least about 95% by weight of Compound 2 is present. In still other embodiments of the invention, at least about 99% by weight of Compound 2 is present.

According to one embodiment, Compound 2 is present in an amount of at least about 97, 97.5, 98.0, 98.5, 99, 99.5, 99.8 weight percent where the percentages are based on the total weight of the composition. According to another embodiment, Compound 2 contains no more than about 3.0 area percent HPLC of total organic impurities and, in certain embodiments, no more than about 1.5 area percent HPLC total organic impurities relative to the total area of the HPLC chromatogram. In other embodiments, Compound 2 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 2 is also meant to include all tautomeric forms of Compound 2. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 2 can exist in a variety of solid forms. Exemplary such forms include polymorphs such as those described herein.

In some embodiments, Compound 2 is amorphous. In some embodiments, Compound 2 is amorphous, and is substantially free of crystalline Compound 2.

In certain embodiments, Compound 2 is a crystalline solid. In other embodiments, Compound 2 is a crystalline solid substantially free of amorphous Compound 2. As used herein, the term “substantially free of amorphous Compound 2” means that the compound contains no significant amount of amorphous Compound 2. In certain embodiments, at least about 95% by weight of crystalline Compound 2 is present. In still other embodiments of the invention, at least about 99% by weight of crystalline Compound 2 is present.

In one embodiment, Compound 2 has a stoichiometry of (Compound 1):(adipic acid) that is about 1:1. The term “mono-adipate” refers to a compound having such stoichiometry.

In another embodiment, Compound 2 has a stoichiometry of (Compound 1):(adipic acid) that is about 2:1. The term “hemi-adipate” refers to a compound having such a stoichiometry.

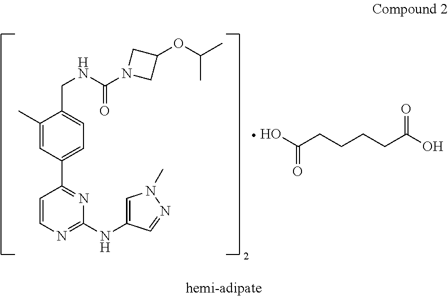

It has been found that Compound 2 can exist in at least two distinct solid forms.

In some embodiments, the stoichiometry of (Compound 1):(adipic acid) is about 1:1. In some embodiments, the present invention provides a solid form of Compound 2 referred to herein as Type I (i.e., “mono-adipate”).

In some embodiments, the stoichiometry of (Compound 1):(adipic acid) is about 2:1. In some embodiments, the present invention provides a solid form of Compound 2 referred to herein as Type II (i.e., “hemi-adipate”).

In the combination of an acid and a base compound for the preparation of a solid form, a Δ pKₐ (pKₐ(base)-pKₐ(acid))≤1 generally will permit the formation of a salt compound where the two compounds are ionized. Where this threshold is not met, non-ionic interactions (e.g., hydrogen bonds) can still occur between neutral acid and the base compounds to form, e.g., a co-crystal. The pKₐ of Compound 1 (the base) was determined to be 3.31 (±0.06) via potentiometric titration whereas adipic acid has a pKₐ₁ of about 4.4 and a pKₐ₂ of about 5.4.

In some embodiments, Compound 2 (e.g. Compound 2 Type I or Compound 2 Type II), is a co-crystal. A “co-crystal” as used herein is a solid that is a crystalline material composed of two or more (e.g., two) molecules in the same crystal lattice. In a co-crystal, hydrogen bonding or other non-covalent or non-ionic molecular interactions (e.g., van der Waals forces or π-π interactions) may exist between the compounds present in the crystal lattice.

### Compound 2 Type I

In some embodiments, Compound 2 Type I has at least 1, 2, 3, 4 or 5 spectral peak(s) selected from the peaks listed in Table 1 below.

TABLE 1
XRPD Peak Positions for Compound 2 Type I¹
Position (°2θ)

6.34
9.24
12.78
15.43
18.26
19.11
19.56
20.57
21.76
22.58
24.01
26.25
27.37
¹In this and all subsequent tables, the position 2θ is within ± 0.2.

In some embodiments, Compound 2 Type I is characterized in that it has one or more peaks in its X-ray powder diffraction pattern selected from those at about 6.34, 9.24, 27.37. In some embodiments, Compound 2 Type I is characterized in that it has two or more peaks in its X-ray powder diffraction pattern selected from those at about 6.34, 9.24, 27.37. In some embodiments, Compound 2 Type I is characterized in that it has all three peaks in its X-ray powder diffraction pattern selected from those at about 6.34, 9.24, 27.37. As used herein, the term “about,” when used in reference to a degree 2-theta value refers to the stated value±0.2 degree 2-theta.

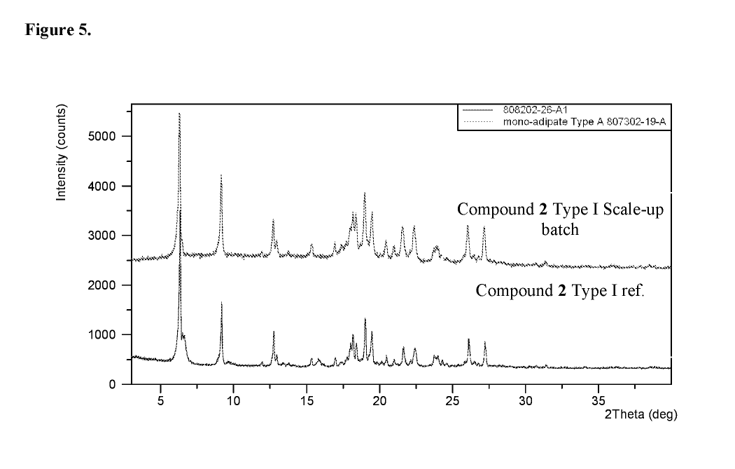

In certain embodiments, the X-ray powder diffraction pattern is substantially similar to the XRPD provided in FIG. 5.

Methods for preparing Compound 2 Type I are described infra.

In some embodiments, the stoichiometry of (Compound 1):(adipic acid) of Compound 2 Type I is about 1:1.

### Compound 2 Type II

In some embodiments, Compound 2 Type II has at least 1, 2, 3, 4 or 5 spectral peak(s) selected from the peaks listed in Table 2 below.

TABLE 2
XRPD Peak Positions for Compound 2 Type II
Position (°2θ)

7.04
8.79
11.17
12.72
13.08
14.12
17.65
18.48
19.38
20.08
21.23
22.43
23.43
24.58
25.14
25.72
27.17
28.48
¹ In this and all subsequent tables, the position 2θ is within ± 0.2.

In some embodiments, Compound 2 Type II is characterized in that it has one or more peaks in its X-ray powder diffraction pattern selected from those at about 7.04, 20.08, 25.14. In some embodiments, Compound 2 Type II is characterized in that it has two or more peaks in its X-ray powder diffraction pattern selected from those at about 7.04, 20.08, 25.14. In some embodiments, Compound 2 Type II is characterized in that it has all three peaks in its X-ray powder diffraction pattern selected from those at about 7.04, 20.08, 25.14.

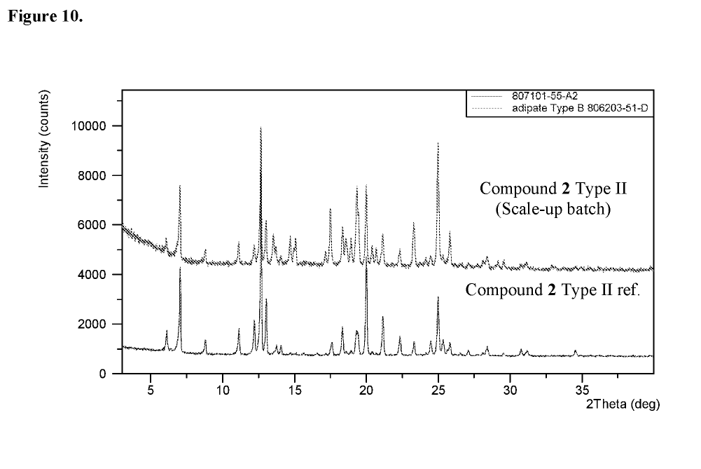

In certain embodiments, the X-ray powder diffraction pattern is substantially similar to the XRPD provided in FIG. 10.

Methods for preparing Compound 2 Type II are described infra.

In some embodiments, the stoichiometry of (Compound 1):(adipic acid) of Compound 2 Type II is about 2:1.

### General Methods of Providing the Compounds

Compound 1 is prepared according to the methods described in detail in the '853 application, the entirety of which is hereby incorporated herein by reference.

As described herein, Compound 2 and forms thereof, are prepared from Compound 1 by combining Compound 1 with adipic acid to form the product Compound 2. The stoichiometry of Compound 1 and adipic acid can be varied. Thus, another aspect of the present invention provides a method for preparing Compound 2, and forms thereof.

As described generally above, in some embodiments, the present invention provides a method for preparing Compound 2:

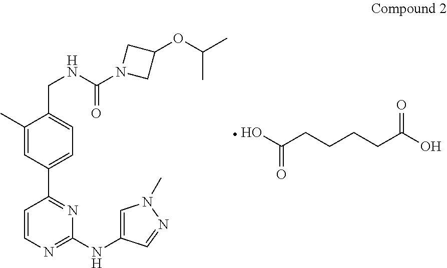 [SMILES: Cc1cc(-c2ccnc(Nc3cnn(C)c3)n2)ccc1CNC(=O)N1CC(OC(C)C)C1 ; O=C(O)CCCCC(=O)O]

comprising steps of:

combining Compound 1:

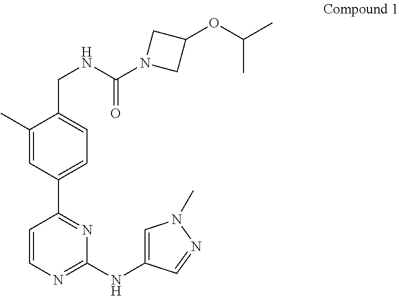 [SMILES: Cc1cc(-c2ccnc(Nc3cnn(C)c3)n2)ccc1CNC(=O)N1CC(OC(C)C)C1]

with adipic acid and optionally a suitable solvent under conditions suitable for forming a Compound 2.

In some embodiments, the present invention provides a method of making a solid form comprising Compound 1 and adipic acid that is Compound 2 Type I.

In some embodiments, the present invention provides a method of making a solid form comprising Compound 1 and adipic acid that is Compound 2 Type II.

In some embodiments, the present invention provides a method of making a solid form comprising Compound 1 and adipic acid that is amorphous.

A suitable solvent may be any solvent system (e.g., one solvent or a mixture of solvents) in which Compound 1 and/or adipic acid are soluble, or are at least partially soluble.

Examples of suitable solvents useful in the present invention include, but are not limited to protic solvents, aprotic solvents, polar aprotic solvent, or mixtures thereof. In certain embodiments, suitable solvents include an ether, an ester, an alcohol, a ketone, or a mixture thereof. In some embodiments, a solvent is one or more organic alcohols. In some embodiments, a solvent is chlorinated. In some embodiments, a solvent is an aromatic solvent.

In certain embodiments, a suitable solvent is methanol, ethanol, isopropanol, or acetone wherein said solvent is anhydrous or in combination with water or heptane. In some embodiments, suitable solvents include tetrahydrofuran, dimethylformamide, dimethylsulfoxide, glyme, diglyme, methyl t-butyl ether, t-butanol, n-butanol, and acetonitrile. In some embodiments, a suitable solvent is ethanol. In some embodiments, a suitable solvent is anhydrous ethanol. In some embodiments, a suitable solvent is MTBE.

In some embodiments, a suitable solvent is ethyl acetate. In some embodiments, a suitable solvent is a mixture of methanol and methylene chloride. In some embodiments, a suitable solvent is a mixture of acetonitrile and water. In certain embodiments, a suitable solvent is methyl acetate, isopropyl acetate, acetone, or tetrahydrofuran. In certain embodiments, a suitable solvent is diethylether. In certain embodiments, a suitable solvent is water. In certain embodiments, a suitable solvent is methyl ethyl ketone. In certain embodiments, a suitable solvent is toluene. In some embodiments, a suitable solvent is tetrahydrofuran.

In some embodiments, the present invention provides a method for preparing Compound 2, comprising steps of removing a solvent and/or adding a solvent. In some embodiments, an added solvent is the same as a solvent removed. In some embodiments, an added solvent is different from a solvent removed. Means of solvent removal are known in the synthetic and chemical arts and include, but are not limited to, any of those described herein and in the ensuing Examples.

In some embodiments, a method for preparing Compound 2 comprises steps of heating and/or cooling a preparation.

In some embodiments, a method for preparing Compound 2 comprises steps of agitating and/or stirring a preparation.

In some embodiments, a method for preparing Compound 2 comprises a step of adding a suitable acid to a solution or slurry of compound 1.

In some embodiments, a method for preparing Compound 2 comprises a step of heating.

In certain embodiments, Compound 2 precipitates from the mixture. In some embodiments, Compound 2 crystallizes from the mixture. In some embodiments, Compound 2 crystallizes from solution following seeding of the solution (i.e., adding crystals of Compound 2 to the solution).

Compound 2 can precipitate out of the reaction mixture, or be generated by removal of part or all of the solvent through methods such as evaporation, distillation, filtration (ex. nanofiltration, ultrafiltration), reverse osmosis, absorption and reaction, by adding an anti-solvent such as heptane, by cooling or by different combinations of these methods.

As described generally above, Compound 2 is optionally isolated. It will be appreciated that Compound 2 may be isolated by any suitable physical means known to one of ordinary skill in the art. In certain embodiments, precipitated solid Compound 2 is separated from the supernatant by filtration. In other embodiments, precipitated Compound 2 is separated from the supernatant by decanting the supernatant.

In certain embodiments, Compound 2 is separated from the supernatant by filtration.

In certain embodiments, an isolated Compound 2 is dried in air. In other embodiments isolated Compound 2 is dried under reduced pressure, optionally at elevated temperature.

As described herein, Compound 2 can be an amorphous solid. Amorphous solids are well known to one of ordinary skill in the art and can be prepared by various methods such as lyophilization, melting, precipitation (e.g., from supercritical fluid), mechanical treatment (e.g., milling), quench cooling, desolvation, rotary evaporation, precipitation, and spray-drying among others.

### Methods of Use

In certain embodiments, compounds of the present invention (e.g., Compound 2) are for use in medicine. In some embodiments, compounds of the present invention are useful as kinase inhibitors. In certain embodiments, compounds of the present invention are selective inhibitors of Btk. In some embodiments, the present invention provides methods of decreasing Btk enzymatic activity. Such methods include contacting a Btk with an effective amount of a provided compound. Therefore, the present invention further provides methods of inhibiting Btk enzymatic activity by contacting a Btk with a compound of the present invention.

In some embodiments, the present invention provides methods of decreasing Btk enzymatic activity. In some embodiments, such methods include contacting a Btk with an effective amount of a provided compound. Therefore, the present invention further provides methods of inhibiting Btk enzymatic activity by contacting a Btk with a compound of the present invention.

Btk enzymatic activity, as used herein, refers to Btk kinase enzymatic activity. For example, where Btk enzymatic activity is decreased, PIP3 binding and/or phosphorylation of PLCγ is decreased. In some embodiments, the half maximal inhibitory concentration (IC₅₀) of a provided compound against Btk is less than 1 uM. In some embodiments, the IC₅₀ of a provided compound against Btk is less than 500 nM. In some embodiments, the IC₅₀ of a provided compound against Btk is less than 100 nM. In some embodiments, the IC₅₀ of a provided compound against Btk is less than 10 nM. In some embodiments, the IC₅₀ of a provided compound against Btk is less than 1 nM. In some embodiments, the IC₅₀ of a provided compound against Btk is from 0.1 nM to 10 uM. In some embodiments, the IC₅₀ of a provided compound against Btk is from 0.1 nM to 1 uM. In some embodiments, the IC₅₀ of a provided compound against Btk is from 0.1 nM to 100 nM. In some embodiments, the IC₅₀ of a provided compound against Btk is from 0.1 nM to 10 nM.

In some embodiments, provided compounds are useful for the treatment of diseases and disorders that may be alleviated by inhibiting (i.e., decreasing) Btk enzymatic activity. By “diseases” is meant diseases or disease symptoms. Thus, the present invention provides methods of treating autoimmune disorders, inflammatory disorders, and cancers in a subject in need thereof. Such methods include administering to the subject a therapeutically effective amount of a provided compound.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barré syndrome (GBS), Hashimoto's disease, idiopathic thrombocytopenic purpura, lupus erythematosus, mixed connective tissue disease, multiple sclerosis, myasthenia gravis, pemphigus vulgaris, pernicious anaemia, polymyositis, primary biliary cirrhosis, Sjögren's syndrome, temporal arteritis, and Wegener's granulomatosis. The term “inflammatory disorders” includes diseases or disorders involving acute or chronic inflammation such as allergies, asthma, prostatitis, glomerulonephritis, pelvic inflammatory disease (PID), inflammatory bowel disease (IBD, e.g., Crohn's disease, ulcerative colitis), reperfusion injury, rheumatoid arthritis, transplant rejection, and vasculitis. In some embodiments, the present invention provides a method of treating rheumatoid arthritis or lupus.

The term “cancer” includes diseases or disorders involving abnormal cell growth and/or proliferation. In some embodiments, such cancers include glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer). In some embodiments, the present invention provides a method of treating leukemia or lymphoma.

The term “subject,” as used herein, refers to a mammal to whom a pharmaceutical composition is administered. Exemplary subjects include humans, as well as veterinary and laboratory animals such as horses, pigs, cattle, dogs, cats, rabbits, rats, mice, and aquatic mammals.

### Assays

To develop useful Tec kinase (e.g., BTK) family inhibitors, candidate inhibitors capable of decreasing Tec kinase family enzymatic activity may be identified in vitro. The activity of the inhibitor compounds can be assayed utilizing methods known in the art and/or those methods presented herein.

Compounds that decrease Tec kinase family members' enzymatic activity may be identified and tested using a biologically active Tec kinase family member, either recombinant or naturally occurring. Tec kinases can be found in native cells, isolated in vitro, or co-expressed or expressed in a cell. Measuring the reduction in the Tec kinase family member (e.g., BTK) enzymatic activity in the presence of an inhibitor relative to the activity in the absence of the inhibitor may be performed using a variety of methods known in the art, such as the POLYGAT-LS assays described below in the Examples. Other methods for assaying the activity of Btk and other Tec kinases are known in the art. The selection of appropriate assay methods is well within the capabilities of those of skill in the art.

Once compounds are identified that are capable of reducing Tec kinase family members' enzymatic activity, the compounds may be further tested for their ability to selectively inhibit a Tec kinase family member relative to other enzymes. Inhibition by a compound of the invention is measured using standard in vitro or in vivo assays such as those well known in the art or as otherwise described herein.

Compounds may be further tested in cell models or animal models for their ability to cause a detectable changes in phenotype related to a Tec kinase family member (e.g., BTK) activity. In addition to cell cultures, animal models may be used to test Tec kinase family member inhibitors for their ability to treat autoimmune disorders, inflammatory disorders, or cancer in an animal model.

### Pharmaceutical Compositions

In another aspect, the present invention provides pharmaceutical compositions comprising Compound 2 or comprising Compound 2 in combination with a pharmaceutically acceptable excipient (e.g., a carrier).

The pharmaceutical compositions include optical isomers, diastereomers, or pharmaceutically acceptable salts of the inhibitors disclosed herein. Compound 2 included in the pharmaceutical composition may be covalently attached to a carrier moiety, as described above. Alternatively, Compound 2 included in the pharmaceutical composition is not covalently linked to a carrier moiety.

A “pharmaceutically acceptable carrier,” as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

The compounds of the invention can be administered alone or can be coadministered to the subject. Coadministration is meant to include simultaneous or sequential administration of the compounds individually or in combination (more than one compound). The preparations can also be combined, when desired, with other active substances (e.g. to reduce metabolic degradation).

### Formulations

Compounds of the present invention can be prepared and administered in a wide variety of oral, parenteral, and topical dosage forms. Thus, the compounds of the present invention can be administered by injection (e.g. intravenously, intramuscularly, intracutaneously, subcutaneously, intraduodenally, or intraperitoneally). Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds of the invention can also be incorporated into liposomes or administered via transdermal pumps or patches. Pharmaceutical admixtures suitable for use in the present invention include those described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

### Effective Dosages

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat cancer, such compositions will contain an amount of active ingredient effective to achieve the desired result (e.g. decreasing the number of cancer cells in a subject).

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the disease responsive to Btk inhibition); presence of other diseases or other health-related problems; kind of concurrent treatment; and complications from any disease or treatment regimen. Other therapeutic regimens or agents can be used in conjunction with the methods and compounds of the invention.

For any compound described herein, the therapeutically effective amount can be initially determined from cell culture assays. Target concentrations will be those concentrations of active compound(s) that are capable of decreasing kinase enzymatic activity as measured, for example, using the methods described.

Therapeutically effective amounts for use in humans may be determined from animal models. For example, a dose for humans can be formulated to achieve a concentration that has been found to be effective in animals. The dosage in humans can be adjusted by monitoring kinase inhibition and adjusting the dosage upwards or downwards, as described above.

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. In come embodiments, the dosage range is 0.001% to 10% w/v. In some embodiments, the dosage range is 0.1% to 5% w/v. Dosage amounts and intervals can be adjusted individually to provide levels of the administered compound effective for the particular clinical indication being treated. This will provide a therapeutic regimen that is commensurate with the severity of the individual's disease state.

### EXAMPLES

The examples below are meant to illustrate certain embodiments of the invention, and not to limit the scope of the invention.

### General Experimental

### Abbreviations

MeOH Methanol

DMSO Dimethyl sulfoxide

NMP 1-Methyl-2-pyrrolidone

DMAc N,N-Dimethyl acetamide

EtOH Ethanol

IPA Isopropyl alcohol

ACN Acetonitrile

DCM Dichloromethane

THF Tetrahydrofuran

2-MeTHF 2-Methyltetrahydrofuran

CHCl₃ Trichloromethane

MIBK Methyl isobutyl ketone

EtOAc Ethyl acetate

IPAc Isopropyl acetate

MTBE Methyl tert-butyl ether

DSC Differential scanning calorimetry

IC Ion chromatography

NMR Nuclear magnetic resonance

TGA Thermogravimetric analysis

XRPD X-ray powder diffraction

### Instruments and Methods

### A. X-Ray Powder Diffraction (XRPD)

For XRPD analysis, a PANalytical Empyrean X-ray powder diffractometer was used. The parameters used are listed in Table 3.

TABLE 3
XRPD Parameters

| Parameter | Value |
|---|---|
| X-Ray wavelength | Cu, kα, Kα1 (Å): 1.540598, Kα2 (Å): 1.544426 |
| | Kα2/Kα1 intensity ratio: 0.50 |
| X-Ray tube setting | 45 kV, 40 mA |
| Divergence slit | Automatic |
| Scan mode | Continuous |
| Scan range (°2TH) | 3°-40° |
| Step size (°2TH) | 0.013 |
| Scan speed (°/min) | About 10 |

### B. Thermogravimetric (TGA) and Differential Scanning Calorimetry (DSC)

TGA data were collected using a TA Q500/Q5000 TGA from TA Instruments. DSC was performed using a TA Q200/Q2000 DSC from TA Instruments. Detailed parameters used are listed in Table 4.

TABLE 4
TGA and DSC Parameters

| Parameters | TGA | DSC |
|---|---|---|
| Method | Ramp | Ramp |
| Sample pan | Platinum, open | Aluminum, crimped |
| Temperature | RT-300° C. | RT-250° C. |
| Heating rate | 10° C./min | 10° C./min |
| Purge gas | N₂ | N₂ |

### C. HPLC

Agilent 1100 with DAD detector was used and detailed chromatographic conditions are listed in Table 5.

TABLE 5
Chromatographic conditions and parameters

| HPLC | Agilent 1100 with DAD detector | |
|---|---|---|
| Column | Waters Xbridge C18 150 × 4.6 mm, 5 μm | |
| Mobile phase | A: 0.1% TFA in H₂O | |
| | B: 0.1% TFA in acetonitrile | |
| Gradient table | Time (min) | % B |
| | 0 | 40 |
| | 4 | 60 |
| | 6 | 60 |
| | 6.1 | 40 |
| | 8 | 40 |

| | |
|---|---|
| Run time | 8 min |
| Post time | 0 min |
| Flow rate | 1.0 mL/min |
| Injection volume | 10 μL |
| Detector wavelength | UV at 300 nm, reference500 nm |
| Column temperature | 40° C. |
| Sampler temperature | RT |
| Diluent | MeOH |

### D. DVS

DVS was measured via a SMS (Surface Measurement Systems) DVS Intrinsic. The relative humidity at 25° C. were calibrated against deliquescence point of LiCl, Mg(NO₃)₂ and KCl. Actual parameters for DVS test were listed in Table 6.

TABLE 6
Parameters for DVS test

| Parameters | DVS |
|---|---|
| Temperature | 25° C. |
| Sample size | 10~20 mg |
| Gas and flow rate | N₂, 200 mL/min |
| dm/dt | 0.002%/min |
| Min. dm/dt stability duration | 10 min |
| Max. equilibrium time | 180 min |
| RH range | 0% RH to 95% RH |
| RH step size | 10% RH from 0% RH to 90% RH |
| | 5% RH from 90% RH to 95% RH |

### E. Solution NMR

Solution NMR was collected on Bruker 400M NMR Spectrometer using DMSO-d₆.

### Example 1

### 3-isopropoxy-N-(2-methyl-4-(2-((1-methyl-1H-pyrazol-4-yl)amino)pyrimidin-4-yl)benzyl)azetidine-1-carboxamide (Compound 1)

The synthesis of Compound 1 is described in detail at Example 21 of the '853 application, which is reproduced herein for ease of reference.

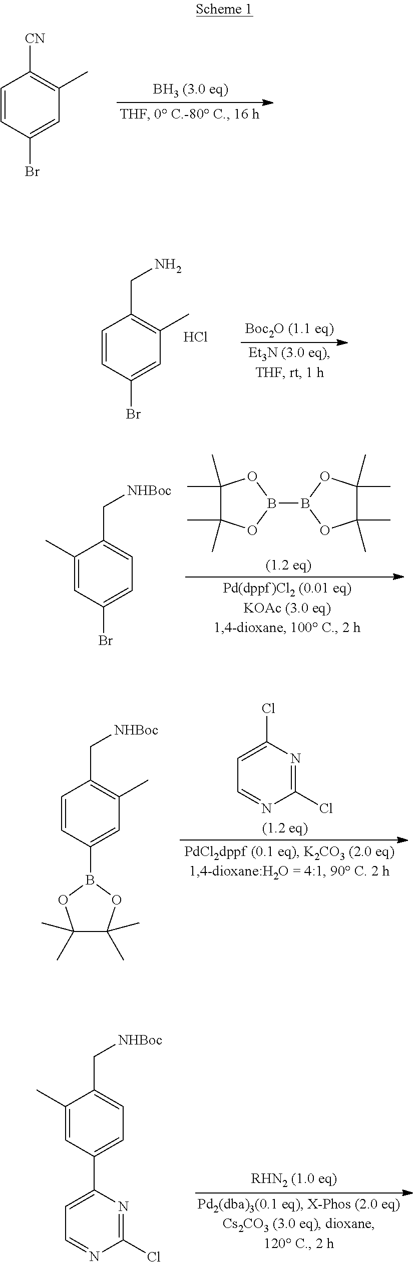

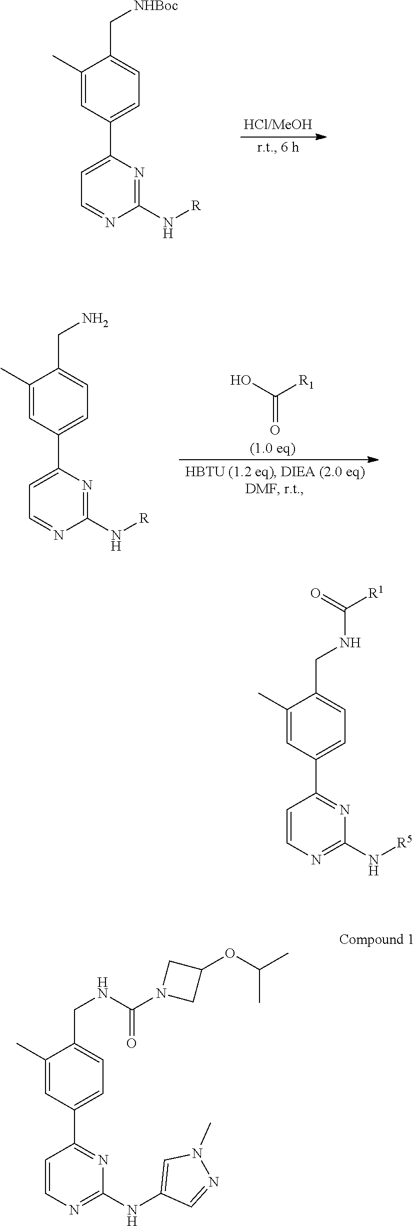

### Preparation of (4-bromo-2-methylphenyl)methanamine

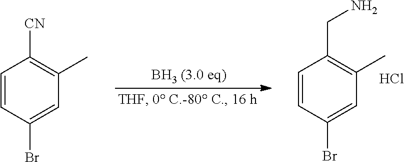

To a solution of 4-bromo-2-methylbenzonitrile (3 g, 15 mmol) in THF (20 mL), BH₃.THF (45 mL, 45 mmol) was added. The solution was stirred at 0° C. for 1 h and heated to 80° C. for 16 h. Then the mixture was quenched with MeOH. After concentrated, the residue was stirred with saturated HCl/EtOAc solution and filtered. The filter cake was rinsed with ether (20 mL ×3) and dried under vacuum to afford (4-bromo-2-methylphenyl)methanamine (3.2 g, yield: 90%) as white solid. ESI-MS (M+H)⁺: 200.1

### Preparation of tert-butyl 4-bromo-2-methylbenzylcarbamate

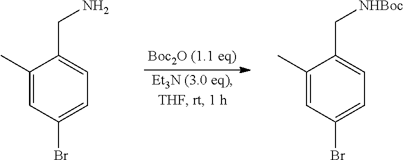

To a solution of (4-bromo-2-methylphenyl)methanamine (1.2 g, 6 mmol) in DCM (30 mL) were added TEA (1.82 g, 18 mmol) and Boc₂O (1.43 g, 6.6 mmol). The mixture was stirred at room temperature for 1 h. After diluted with water (50 mL), the mixture was extracted with DCM (50 mL×2). The combined organics were washed with brine (50 mL), dried (Na₂SO₄), filtered and concentrated to give crude title product (1.7 g, yield 95%) as a white solid, which was used directly in the next step without further purification. ESI-MS (M+H)⁺: 300.1.

### Preparation of tert-butyl 2-methyl-4-(4,4,5,5-tetramethyl-1,3,2-dioxaborolan-2-yl)benzylcarbamate

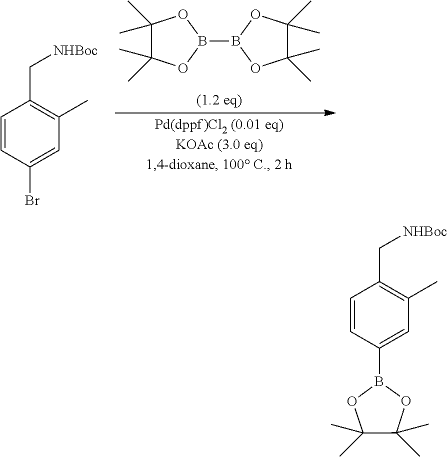

To a solution of tert-butyl 4-bromo-2-methylbenzylcarbamate (1.5 g, 5.0 mmol) in 1,4-dioxane (15 mL) were added 4,4,4′,4′,5,5,5′,5′-octamethyl-2,2′-bi(1,3,2-dioxaborolane) (1.52 g, 6.0 mmol), KOAc (1.75 g, 18 mmol) and Pd(dppf)Cl₂DCM (407 mg, 0.5 mmol) under nitrogen. The mixture was stirred at 100° C. for 2 h. After cooling down to room temperature, the mixture was diluted with water (50 mL) and extracted with ethyl acetate (100 mL×3). The combined organic layer was washed with brine, dried, concentrated and purified by silica gel column (petroleum ether/EtOAc=10:1) to give tert-butyl 2-methyl-4-(4,4,5,5-tetramethyl-1,3,2-dioxaborolan-2-yl)benzylcarbamate (1.2 g, yield 69%) as white solid. ESI-MS (M+H)⁺: 348.2. ¹H NMR (400 MHz, CDCl₃) δ: 7.61-7.59 (m, 2H), 7.26 (s, 1H), 4.68 (br, 1H), 4.33 (d, J=5.6 Hz, 2H), 2.32 (s, 3H), 1.45 (s, 9H), 1.34 (s, 12H).

### Preparation of tert-butyl 4-(2-chloropyrimidin-4-yl)-2-methylbenzylcarbamate

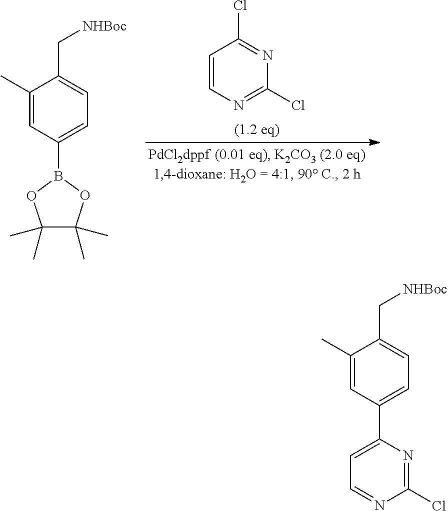

To a solution of tert-butyl 2-methyl-4-(4,4,5,5-tetramethyl-1,3,2-dioxaborolan-2-yl)benzylcarbamate (3.47 g, 10 mmol) and 2,4-dichloropyrimidine (1.79 g, 12 mmol) in 1,4-dioxane (28 mL) and H₂O (7 mL), Pd(dppf)Cl₂DCM (815 mg, 1.0 mmol) and K₂CO₃ (2.76 g, 20 mmol) were added under N₂. The mixture was stirred at 90° C. for 2 h. After cooling to room temperature, the mixture was diluted with H₂O (80 mL) and extracted with EA (80 mL×2). The organic layers were dried and concentrated. The residue was purified by column chromatography (silica, petroleum ether/EtOAc=5:1 to 2:1) to give tert-butyl 4-(2-chloropyrimidin-4-yl)-2-methylbenzylcarbamate (2.67 g, yield 80%) as white solid ESI-MS (M+H)⁺: 334.1. ¹H NMR (400 MHz, CDCl₃) δ: 8.12 (d, J=5.2 Hz, 1H), 7.92 (s, 1H), 7.87 (d, J=8.0 Hz, 1H), 7.63 (d, J=5.6 Hz, 1H), 7.40 (d, J=7.6 Hz, 1H), 4.84 (br, 1H), 4.38(d, J=5.2 Hz, 1H), 2.41 (s, 3H), 1.47 (s, 9H).

### Preparation of tert-butyl 2-methyl-4-(2-((1-methyl-1H-pyrazol-4-yl)amino)pyrimidin-4-yl)benzylcarbamate

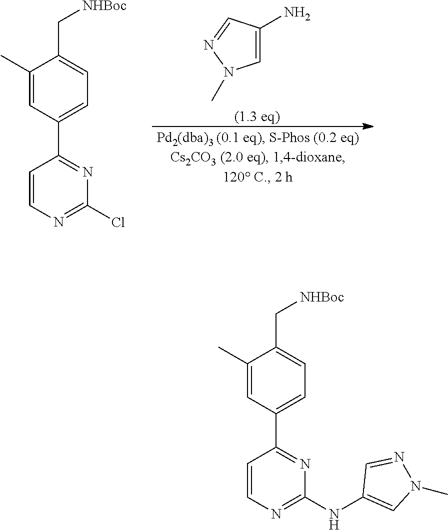

To a solution of tert-butyl 4-(2-chloropyrimidin-4-yl)-2-methylbenzylcarbamate (333 mg, 1.0 mmol) and 1-methyl-pyrazol-4-amine (126 mg, 1.3 mmol) in 1,4-dioxane (5 mL), Pd₂(dba)₃ (92 mg, 0.1 mmol), S-Phos (82 mg, 0.2 mmol) and Cs₂CO₃ (650 mg, 2.0 mmol) were added under N₂. The mixture was stirred at 120° C. for 2 h. After cooling to room temperature, the mixture was diluted with H₂O (40 mL) and extracted with EA (60 mL×2). The organic layers were dried and concentrated. The residue was purified by column chromatography (silica, petroleum ether/EtOAc=3:1 to 1:1) to give tert-butyl 2-methyl-4-(2-((1-methyl-1H-pyrazol-4-yl)amino)pyrimidin-4-yl)benzylcarbamate (248 mg, yield 63%) as white solid ESI-MS (M+H)⁺: 395.1. ¹H NMR (400 MHz, CD₃OD) δ:8.38 (d, J=5.2 Hz, 1H), 7.97-7.93 (m, 3H), 7.65 (s, 1H), 7.38 (d, J=8.0 Hz, 1H), 7.20 (d, J=9.2 Hz, 1H), 4.30 (s, 2H), 3.85 (s, 3H), 2.42 (s, 3H), 1.48 (s, 9H).

### Preparation of 4-(4-(aminomethyl)-3-methylphenyl)-N-(1-methyl-1H-pyrazol-4-yl)pyrimidin-2-amine

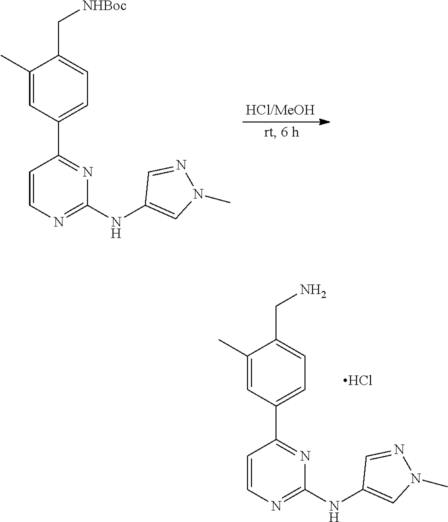

A mixture of tert-butyl 2-methyl-4-(2-((1-methyl-1H-pyrazol-4-yl)amino)pyrimidin-4-yl)benzylcarbamate (3.94 g, 10.0 mmol) in a solution of HCl in methanol (30 mL, prepared from gas HCl) was stirred at room temperature for 6 h. The solvent was removed and the solid was rinsed with cold diethyl ether (100 mL). The solid was dried under vacuum to give 4-(4-(aminomethyl)-3-methylphenyl)-N-(1-methyl-1H-pyrazol-4-yl)pyrimidin-2-amine (2.97 g, yield 90%) as a yellow solid ESI-MS (M+H)⁺: 295.1. ¹H NMR (400 MHz, D₂O) δ: 7.98-7.96 (m, 1H), 7.66-7.22 (m, 6H), 4.10 (s, 2H), 3.68 (s, 3H), 2.20 (s, 3H).

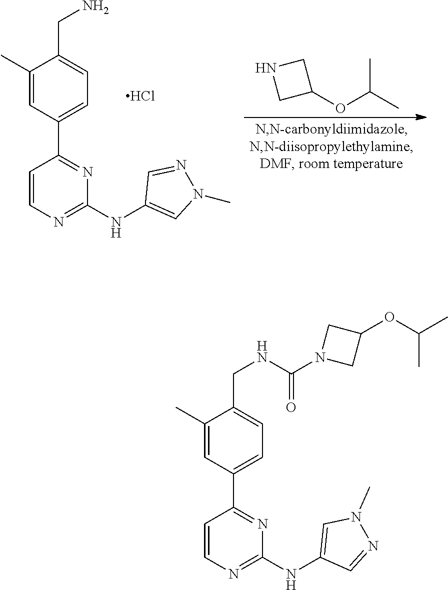

4-(4-(aminomethyl)-3-methylphenyl)-N-(1-methyl-1H-pyrazol-4-yl)pyrimidin-2-amine hydrochloride (prepared in Example 1) (200 mg, 0.7 mmol), 3-isopropoxy azetidine (113 mg, 0.747mmol), and N,N-carbonyldiimidazole (0.110 g, 0.679 mmol) in N,N-dimethylformamide (1.58 mL, 20.4 mmol) was added N,N-diisopropylethylamine (0.473 mL, 2.72 mmol) slowly and stirred at room temperature overnight. The mixture was filtered through celite and washed with DMF and purified by prep HPLC to give product as a solid (82 mg, yield: 30%). LCMS: Rt=1.05 min, m/z 436.3. 1H NMR (400 MHz, DMSO-d6) δ: 9.48 (s, 1H), 8.45 (d, J=5.02 Hz, 1H), 7.92 (s, 3H), 7.55 (br. s., 1H), 7.35 (d, J=8.53 Hz, 1H), 7.25 (d, J=5.27 Hz, 1H), 6.84 (s, 1H), 4.15-4.48 (m, 3H), 3.90-4.13 (m, 2H), 3.83 (s, 3H), 3.46-3.69 (m, 3H), 2.36 (s, 3H), 1.08 (d, J=6.27 Hz, 6H).

### Example 2

### Initial Additive Screening

Compound 1 was used as the starting material for additive screening for the preparation of new solid forms. The mixtures of Compound 1 and co-formers were stirred at different temperatures depending on the observation after mixing (Table 7):
1. Suspensions with obvious color change indicating possible reactions were stirred at room temperature.
2. Clear solutions were stirred at 4° C. to induce precipitation.
3. Suspensions with undissolved solids and no apparent color change were stirred at 50° C. to speed up the reaction.
4. Clear solutions obtained after stirring were allowed to evaporate slowly at room temperature in order to maximize the chances of identifying as many crystalline hits as possible.

TABLE 7
Experimental details of combination
of Compound 1 with adipic acid

| Solvent | Temperature | Appearance | Solid Forms Obtained |
|---|---|---|---|
| MeOH/H₂O | 4° C. | C | Compound 2 Type I |
| Acetone | 50° C. | P | Compound 1 Type A |
| ACN | 50° C. | P | Compound 1 Type A |
| EtOH | 4° C. | P | Compound 1 Type A |
| THF | 4° C. | C | Adipic Acid + |
| | | | Compound 2 Type I |
| DCM | 4° C. | P | Compound 1 Type B + |
| | | | Compound 2 Type II |

P: precipitates observed.
C: clear solution observed and allowed to evaporate at room temperature.

As shown in Table 7, two solid forms, Compound 2 Type I and Compound 2 Type II, were obtained from screening. The solid forms obtained from these experiments were characterized by TGA and DSC.

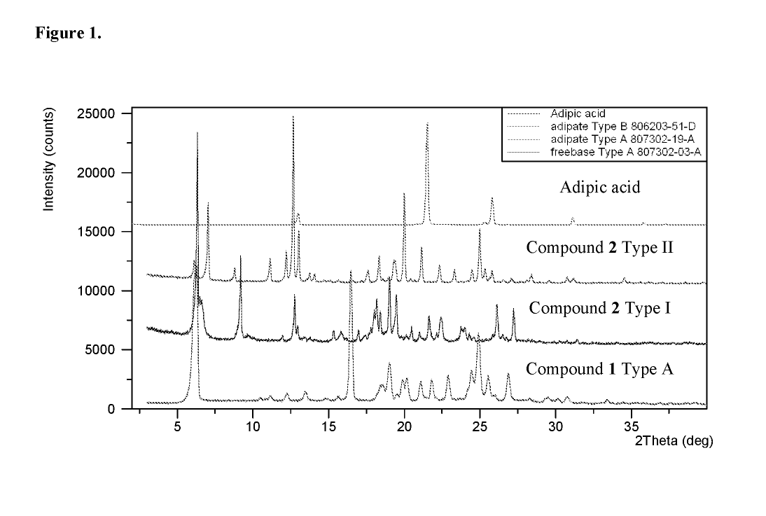

FIG. 1 shows the XRPD patterns of Compound 2 Type I and Compound 2 Type II along with adipic acid and Compound 1 Type A.

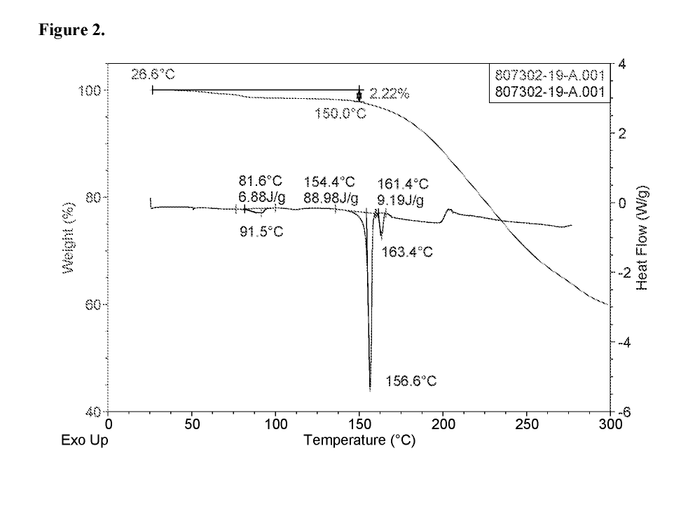

FIG. 2 shows DSC/TGA data of Compound 2 Type I. The figure shows an endotherm at 81.6° C. followed by another endotherm apparently associated with melting/decomposition at 154.4° C. (onset temperature), right after which a third endotherm appeared. A weight loss of 2.2% up to 150° C. was observed.

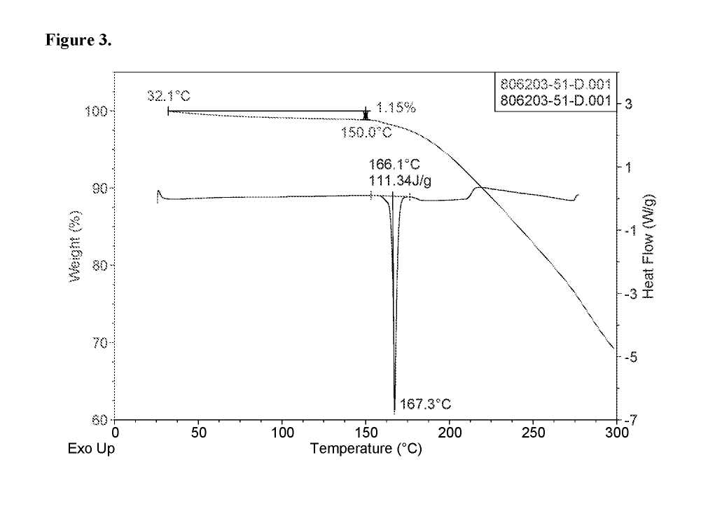

FIG. 3 shows DSC/TGA data of Compound 2 Type II. The figures shows an endotherm apparently associated with melting/decomposition at 166.1° C. (onset temperature). A weight loss of 1.2% up to 150° C. was observed.

### Example 3

### Scale-Up Preparation of Compound 2 Type II

The preparation of Compound 2 Type II was scaled up using the following procedure.
1. 43 mL of THF and 1.0 g Compound 1 were added into a 100-mL vessel.
2. The suspension was heated to 45° C. with agitation (500 rpm).
3. 0.35 g of adipic acid was added to the vessel.
4. The reaction was stirred at 45° C. until a clear solution is obtained.
5. 42 mL of n-heptane (anti-solvent) was added to induce precipitation.
6. The suspension was cooled to room temperature slowly within 1 hr.
7. 43.4 mL of n-heptane was added gradually within 5 hrs and then the mixture was stirred at room temperature for 12 hrs.
8. The mixture was vacuum filter and the wet cake was dried under vacuum at 50° C.
9. Solids were collected (1.3 g obtained, with yield of ˜96.3%).

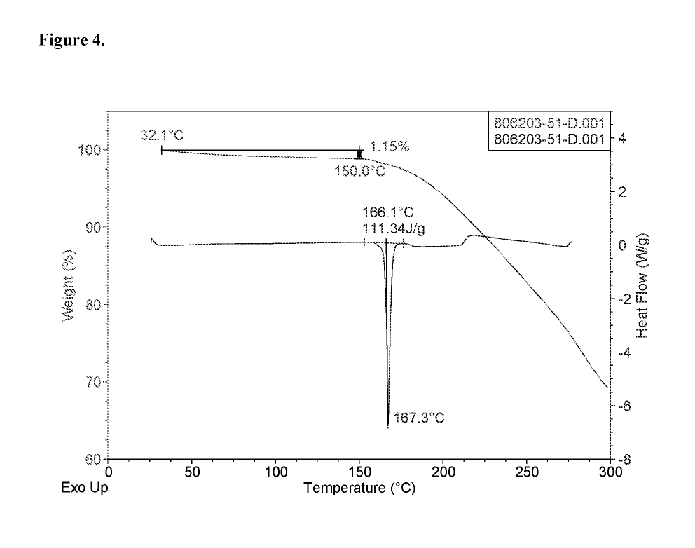

FIG. 4 shows TGA/DSC data for Compound 2 Type II. The data showed a weight loss of 1.2% up to 150° C. and an apparent melting/decomposition endotherm at 166.1° C. (onset temperature).

### Example 4

### Scale-Up Preparation of Compound 2 Type I (Procedure 1)

The preparation of Compound 2 Type I was scaled-up according to the following procedure.
1. 80 mg of Compound 1 and 80 mg of adipic acid (charge molar ratio of 1:3, Compound 1/acid) were weighed into a 20-mL glass vial.
2. 5 mL THF was added to the vial and the mixture was stirred at 50° C. to get a clear solution.
3. The solution was poured totally into 8 mL of n-heptane to induce precipitation. The mixture was stirred at room temperature, and a gel was first observed.
4. After agitation for about 5 minutes, yellow solids were generated and stirred for another 2 hrs.
5. The mixture was vacuum filtered and the cake transferred to dry at 50° C. for 2 hrs. Collect solids for characterization.

FIG. 5 provides the XRPD pattern for the scale-up sample, which conformed to previously-prepared Compound 2 Type I.

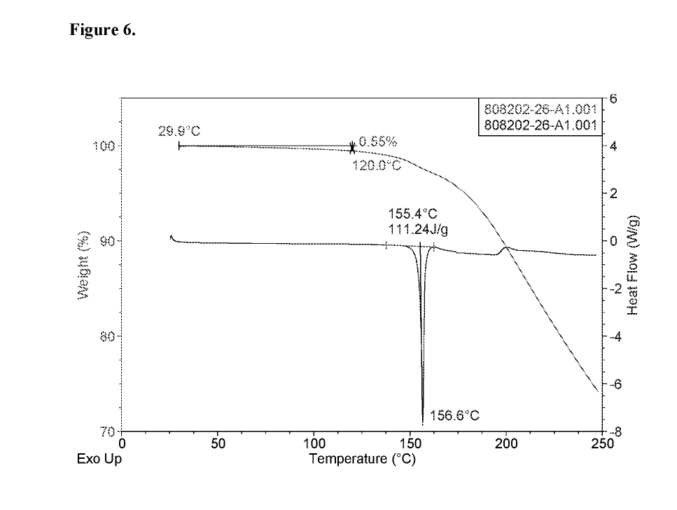

FIG. 6 provides TGA and DSC data for Compound 2 Type I obtained by the scale-up procedure. A weight loss of 0.6% was observed up to 120° C. in TGA, and DSC result showed a sharp melting endotherm at 155.4° C. (onset temperature), suggesting that Compound 2 Type I is an anhydrate.

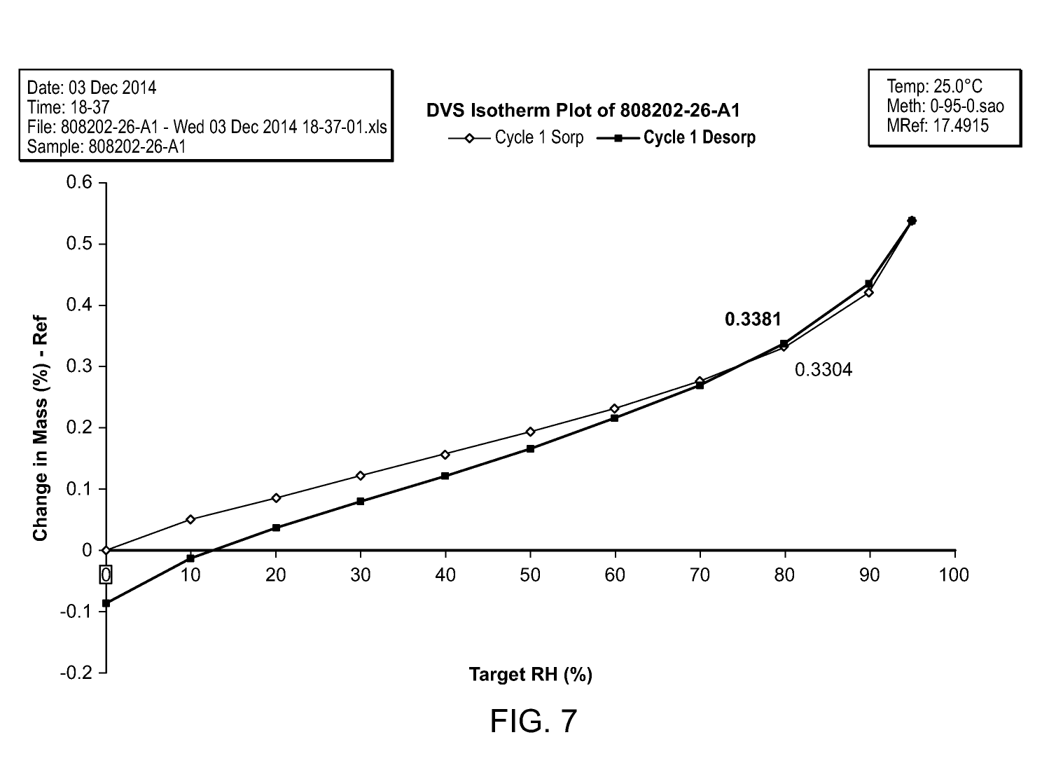

FIG. 7 provides the DVS result showing a water uptake of 0.3% at 25° C./80% RH, indicating Compound 2 Type I is slightly hygroscopic.

Further, XRPD data showed that no form change was observed after the DVS test.

### Example 5

### Scale-Up Preparation of Compound 2 Type I (Procedure 2)

The preparation of Compound 2 Type I on a three-gram scale followed the below procedure.
1. 2.4 g of Compound 1 and 3.1 g of adipic acid (charge molar ratio of 1:4, freebase/acid) were weighed into a 500-mL crystallizer.
2. 116 mL THF was added and the mixture stirred at 50° C. to obtain a clear solution.
3. 58 mL n-heptane was charged into the solution followed by addition of 120 mg of Compound 2 Type I seed. Cloudy material was generated immediately.
4. 116 mL n-heptane was continued to charge and the system oiled out.
5. The reaction was cooled to room temperature and stirred for 18 hrs. The reaction was sampled and analyzed by XRPD and DSC, indicating excess acid was generated.
6. 20 mL THF was added to the crystallizer and stirred for 2 hrs. The reaction was sampled and analyzed by XRPD and DSC, indicating pure Compound 2 Type I was obtained.
7. The reaction was filed and then vacuum dried at room temperature for 1 hr.
8. Solids were collected: 3.2 g (productivity, ˜99%).

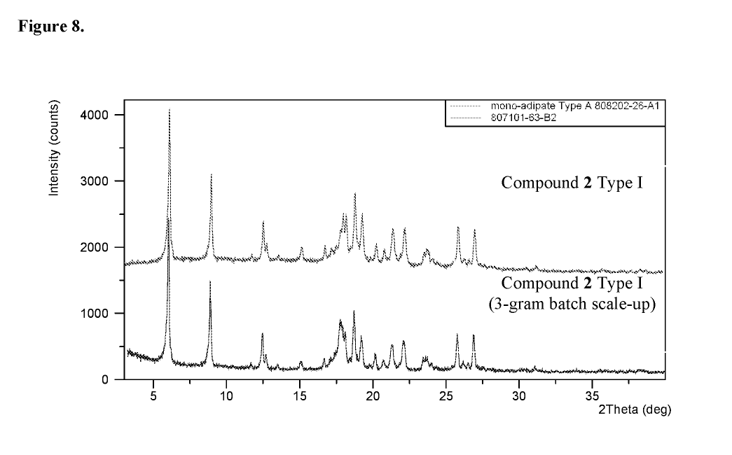

FIG. 8 shows the XRPD pattern of Compound 2 Type I prepared using this method and indicates that the desired product was successfully prepared.

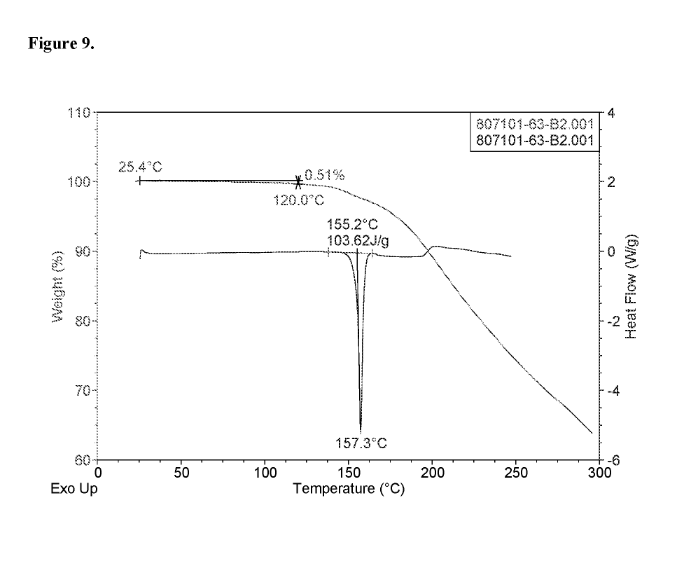

FIG. 9 shows TGA and DSC data Compound 2 Type I prepared using this method. In this figure, a weight loss of 0.5% up to 120° C. was observed in TGA and DSC result showed a sharp melting endotherm at 155.2° C. (onset temperature).

### Example 6

### Preparation of Compound 2 Type II

Compound 2 Type II was prepared according to the below procedure.
1. 150 mL THF and 3.5 g of Compound 1 was added into a 500-mL crystallizer at 45° C. to obtain a clear solution, followed by the addition of 1.24 g of adipic acid.
2. 100 mL n-heptane and 0.88 g seed compound was added to the crystallizer, followed by addition of 200 mL n-heptane over 5 hrs.
3. The batch was cooled to 5° C. and kept at 5° C. for 1.5 hrs.
4. The batch was filtered and then vacuumed dry at room temperature for 2hrs.
5. Collect solids 3.7 g (productivity, ˜92%).

FIG. 10 shows the XRPD pattern for the Compound 2 Type II obtained by the scale-up preparation.

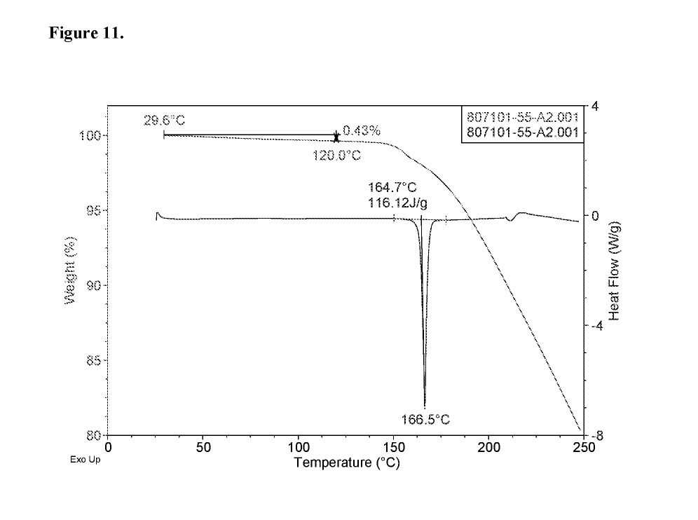

FIG. 11 shows the TGA and DSC data for Compound 2 Type II. A weight loss of 0.4% was observed up to 120° C. in TGA, and DSC result showed a sharp melting endotherm at 164.7° C. (onset temperature), suggesting that Compound 2 Type II is an anhydrate.

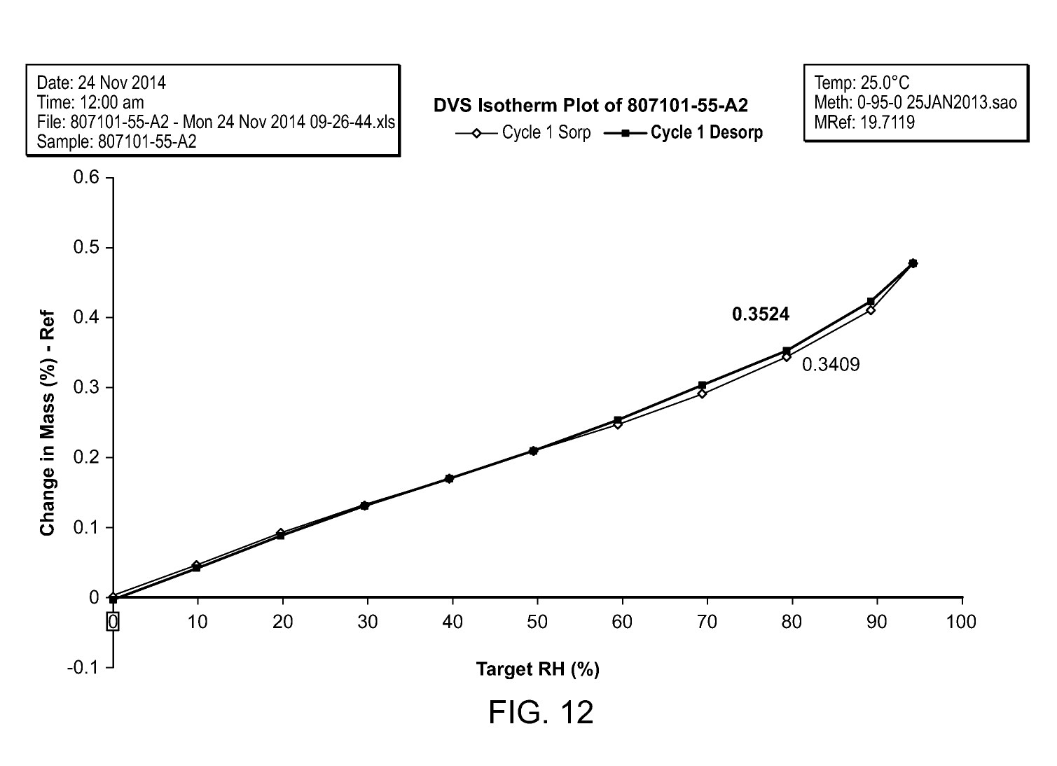

FIG. 12 provides the DVS result, which showed a water uptake of 0.3% at 25 ° C./80% RH, indicating that Compound 2 Type II is slightly hygroscopic.

Further, XRPD data showed that no form change was observed after the DVS test.

### Example 7

### Preparation of Compound 2 Type II (Procedure 2)

Compound 2 Type II was prepared according to the below procedure.

### Preparation of 4-(4-(aminomethyl)-3-methylphenyl)-N-(1-methyl-1H-pyrazol-4-yl)pyrimidin-2-amine

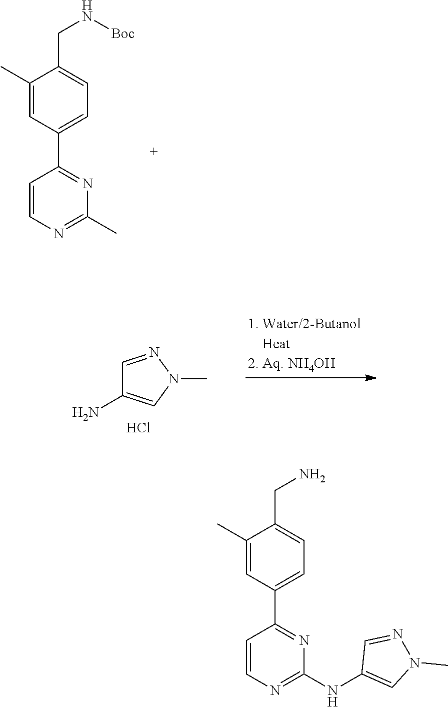 [0202] 1. 5.0 kg of 1-methyl-pyrazol-4-amine hydrochloride and 9.2 kg of water were added to a flask (“Flask 1”).
2. The contents of Flask 1 were stirred at 15-25° C. until dissolved.
3. 9.6 kg of tert-butyl 4-(2-chloropyrimidin-4-yl)-2-methylbenzylcarbamate, 15.6 kg of 2-butanol, and 14.6 kg of water were added to a separate flask (“Flask 2”).
4. The contents of Flask 2 were stirred at 55-65° C. until a clear solution was observed.
5. The contents of Flask 1 were added to Flask 2, rinsing Flask 1 with 5 kg of water and transferring the rinse to Flask 2.
6. The contents of Flask 2 were heated to 80-90° C. and were stirred at 80-90° C. for at least 16 hours.
7. The contents of Flask 2 were cooled to 30-40° C.
8. 46.1 g of water was added to Flask 2 while maintaining the temperature at 30-40° C.
9. 24.2 kg of ammonium hydroxide (28-30%) was diluted with 25.7 kg of water and added to Flask 2 over at least 1 hour.
10. 5 kg of water was added to Flask 2.
11. The contents of Flask 2 were cooled to 10-20° C. over at least 1 hour.
12. The contents of Flask 2 were stirred at 10-20° C. for at least 1 hour.
13. The contents of Flask 2 were vacuum filtered to isolate a solid product.
14. The product was dried under vacuum at ≤75° C.
15. 7.65 kg of product was obtained (90.0% yield), and NMR conformed to prior assignments.

### Preparation of Compound 1

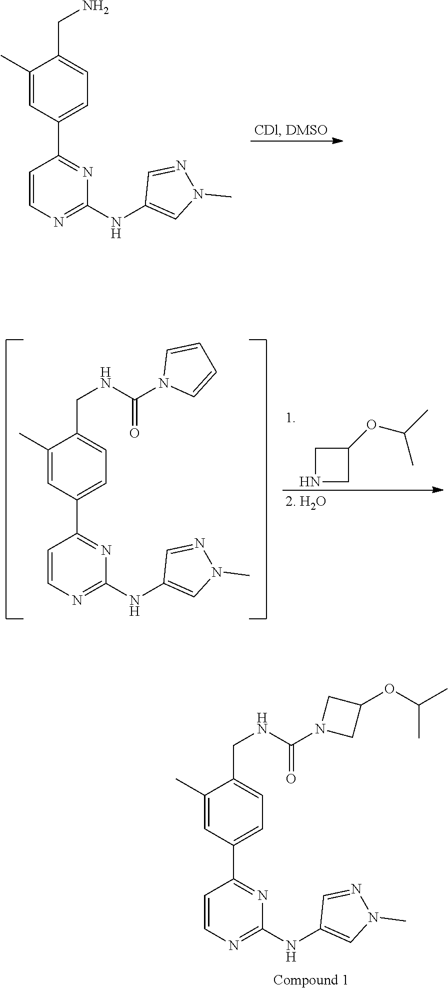 [0218] 1. 22.0 kg of dimethylsulfoxide (DMSO) and 6.6 kg of 4-(4-(aminomethyl)-3-methylphenyl)-N-(1-methyl-1H-pyrazol-4-yl)pyrimidin-2-amine were added to a flask (“Flask 3”).
2. The contents of Flask 3 were stirred at 28-32° C. until solids were dissolved.
3. 22.0 kg of DMSO was added to a separate flask (“Flask 4”).
4. 4.4 kg of 1,1′-carbonyldiimidazole (CDI) was added to Flask 4.
5. The contents of Flask 4 were stirred at 28-32° C. until solids were dissolved.
6. The contents of Flask 3 were added into Flask 4 over at least 1 hour at 28-32° C., rinsing Flask 3 with 2.1 kg DMSO and transferring the rinse to Flask 4.
7. The contents of Flask 4 was stirred at 28-32° C. for at least 30 minutes.
8. 3.4 kg of 3-isopropoxyazetidine was added to Flask 4, and the transfer container and line were rinsed with 1 kg of DMSO, adding the rinse to Flask 4.
9. The contents of Flask 4 were stirred at 28-32° C. for at least 4 hours.
10. The contents of Flask 4 were cooled to ≤20° C.
11. 23.2 kg of water was added with a temperature between 0-5° C. to Flask 4 at a rate that maintained the temperature of the contents of Flask 4 at ≤30° C.
12. The contents of Flask 4 were cooled to 18-20° C.
13. 0.132 kg of Compound 1 seed crystal were charged to Flask 4.
14. The contents of Flask 4 were stirred for at least 1 hour to precipitate product.
15. 23.1 kg of water were charged to Flask 4 at a rate that limited the temperature to ≤30° C.
16. The contents of Flask 4 were stirred for at least 30 minutes at 15-25° C.
17. The contents of Flask 4 were vacuum filtered to isolate a solid product.
18. The product was washed three times with 19.8 kg of water.
19. The product was transferred to trays and dried under vacuum at ≤60° C. to ≤2% water.
20. 9.7 kg of Compound 1 was obtained (99.0% yield), and NMR conformed to prior assignments.

### Preparation of Compound 2 Type II

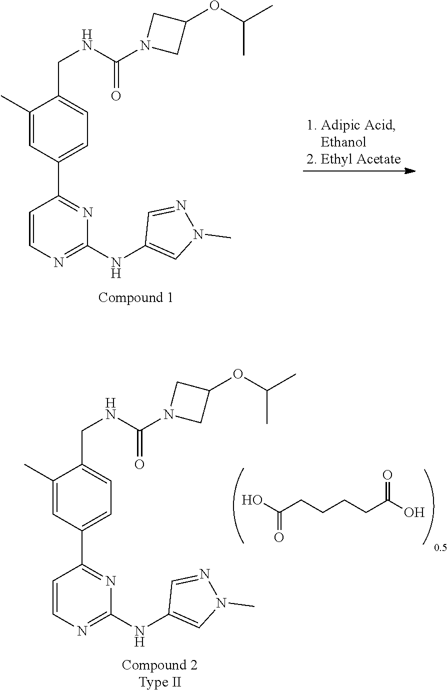 [0239] 1. 18.4 L of ethanol and 4.6 kg of Compound 1 were each added to two flasks (“Flask 5” and “Flask 6”).
2. The contents of Flask 5 and Flask 6 were heated to 70-75° C.
3. 13.8 L of ethanol and 1.4 kg adipic acid were each added to two flasks (“Flask 7” and “Flask 8”).
4. The contents of Flask 7 and Flask 8 were heated to 70-75° C.
5. The contents of Flask 7 were added into Flask 5, rinsing Flask 7 with 2.3 L of ethanol and adding the rinse to Flask 5.
6. The contents of Flask 8 were added into Flask 6, rinsing Flask 8 with 2.3 L of ethanol and adding the rinse to Flask 6.
7. The contents of Flask 5 and Flask 6 were stirred at 70-75° C. for at least 30 minutes.
8. The contents of Flask 5 and Flask 6 were filtered through a 0.45 μm polish filter into a separate flask (“Flask 9”), rinsing Flask 5 and Flask 6 each with 2.3 L of ethanol, adding the rinses to Flask 9.
9. The contents of Flask 9 were agitated at 70-75° C. for at least 30 minutes.
10. The contents of Flask 9 were cooled to 60-64° C.
11. 120 g of Compound 2 Type II and 5 L of ethanol were added to a flask (“Flask 10”).
12. The contents of Flask 10 were stirred at 15-25° C. for at least 2 hours.
13. The contents of Flask 10 were filtered to remove solids and the filtrate reserved (“Flask 10 filtrate”).
14. 184 g of Compound 2 Type II seed was added to a flask (“Flask 11”).
15. 2 L of Flask 10 filtrate was added to Flask 11.
16. The contents of Flask 11 were added to Flask 9, rinsing Flask 11 with 2 L of Flask 10 filtrate and transferring the rinse to Flask 9.
17. The contents of Flask 9 were stirred at 55-65° C. for at least 1 hour.
18. The resultant slurry in Flask 9 was cooled to 0-10° C. over at least 3 hours.
19. The slurry in Flask 9 was stirred at 0-10° C. for at least 30 minutes.
20. The slurry in Flask 9 vacuum was filtered to collect a solid product.
21. The product cake was washed three times with 25 kg polish filtered ethyl acetate. The cake was allowed to soak in the rinse solvent for at least 15 minutes before applying vacuum for each rinse.
22. The product was transferred to trays and dried under vacuum at ≤50° C. to constant weight.
23. 9.65 kg of product was obtained (90% yield); HPLC indicated 99.5% purity.

### Example 8

### Thermodynamic Relationship Investigation via Solubility Measurement

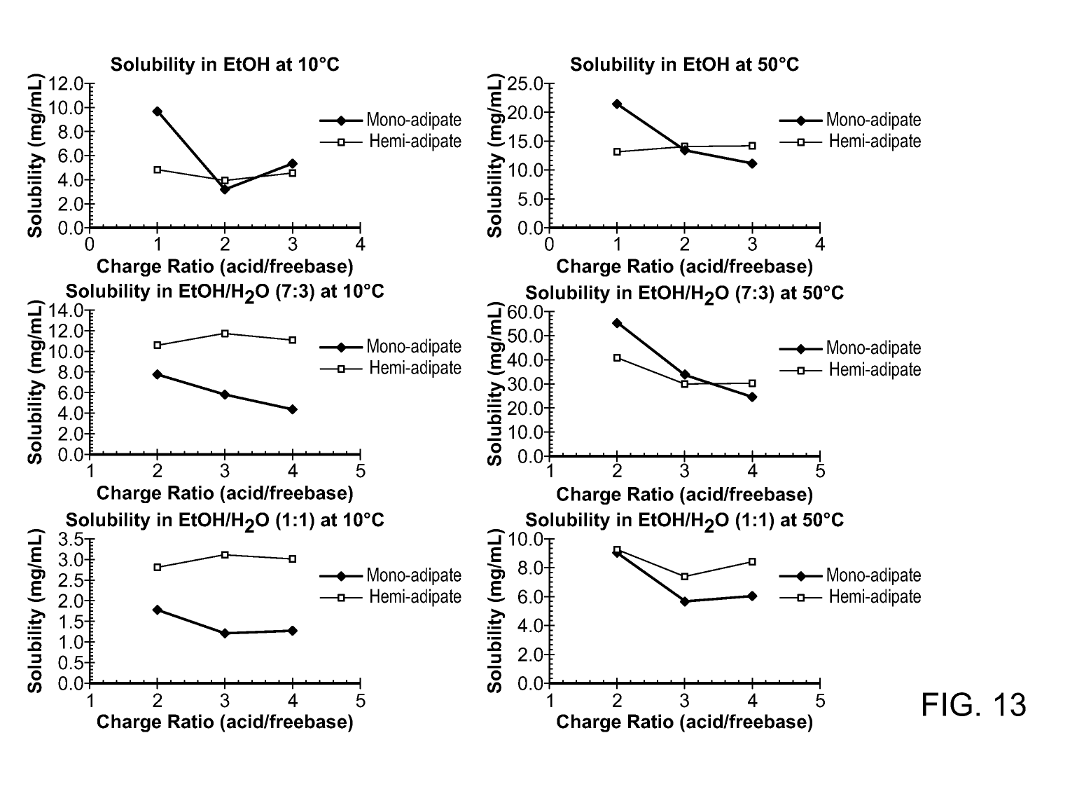

The lead process solvent system is EtOH or co-solvent of EtOH and water. Varying temperature (10 and 50° C.), molar charge ratio of acid to Compound 1 (1, 2, 3 and 4), and solvent ratio of EtOH to water (v/v=1/0, 7/3, and 1/1) were investigated. 70-100 mg of Compound 2 Type I or II were weighed and added with a calculated amount of acid into 1.5-mL glass vial, along with 0.5 mL of corresponding solvent into the vial. The mixture was magnetically stirred under desired conditions for 4 days. The remaining solids were isolated for XRPD analysis and concentrations in filtered mother liquors were measured via HPLC. Solubility data are listed in Table 8 and illustrated in FIG. 13. The results indicated that elevated temperature, low charge ratio of acid to freebase and decreased water content are preferred to stabilize Compound 2 Type II.

TABLE 8
Solubility summary of Compound 2 Types I and II

| Starting | | Temp. | Charge Ratio (acid/freebase) | | | |
|---|---|---|---|---|---|---|
| Form | Solvent (v/v) | (° C.) | 1:1 | 2:1 | 3:1 | 4:1 |
| Com- | EtOH | 10 | 9.7 | 3.2 | 5.3 | — |
| pound 2 | | | mg/mL | mg/mL | mg/mL | |
| Type I | | 50 | 21.5 | 13.4 | 11.2 | — |
| | | | mg/mL | mg/mL | mg/mL | |
| | EtOH/H₂O (7:3) | 10 | — | 7.8 | 5.9 | 4.5 |
| | | | | mg/mL | mg/mL | mg/mL |
| | | 50 | — | 55.9 | 34.3 | 25.1 |
| | | | | mg/mL | mg/mL | mg/mL |
| | EtOH/H₂O (1:1) | 10 | — | 1.8 | 1.2 | 1.3 |
| | | | | mg/mL | mg/mL | mg/mL |
| | | 50 | — | 9.0 | 5.7 | 6.1 |
| | | | | mg/mL | mg/mL | mg/mL |
| Com- | EtOH | 10 | 4.8 | 3.9 | 4.6 | — |
| pound 2 | | | mg/mL | mg/mL | mg/mL | |
| Type II | | 50 | 13.0 | 14.0 | 14.2 | — |
| | | | mg/mL | mg/mL | mg/mL | |
| | EtOH/H₂O (7:3) | 10 | — | 10.6 | 11.7 | 11.1 |
| | | | | mg/mL | mg/mL | mg/mL |
| | | 50 | — | 41.4 | 30.6 | 31.2 |
| | | | | mg/mL | mg/mL | mg/mL |
| | EtOH/H₂O (1:1) | 10 | — | 2.8 | 3.1 | 3.0 |
| | | | | mg/mL | mg/mL | mg/mL |
| | | 50 | — | 9.2 | 7.4 | 8.4 |
| | | | | mg/mL | mg/mL | mg/mL |

—: Relevant experiments were not set up.

### Example 9

### Polymorph Screening of Compound 2 Type II

The solubility of Compound 2 Type II was tested in 20 solvents at room temperature (20±3° C.). Approximately 2 mg solids were added into a 3-mL glass vial. Solvents in Table 9 were then added stepwise (100 μL per step) into the vials until the solids were dissolved or a total volume of 2 mL was reached. Results are summarized in Table 9 and used to guide the solvent selection in polymorph screening.

Polymorph screening of Compound 2 Type II was performed using different solution crystallization or solid transition methods. The methods utilized and crystal forms identified in the screening are summarized in Table 10. As the table shows, no new crystal form of Compound 2 Type II was discovered in the screening.

TABLE 9
Approximate solubility of Compound
2 Type II at room temperature

| Solvent | Solubility (mg/mL) | Solvent | Solubility (mg/mL) |
|---|---|---|---|
| MeOH | S > 19.0 | 2-MeTHF | 6.3 < S < 9.5 |
| EtOH | 10.5 < S < 21.0 | 1,4-Dioxane | 9.5 < S < 19.0 |
| IPA | 5.0 < S < 6.7 | NMP | S > 22.0 |
| ACN | 2.3 < S < 2.6 | DMSO | S > 19.0 |
| Acetone | 5.5 < S < 7.3 | DCM | 4.4 < S < 5.5 |
| MIBK | 1.1 < S < 1.2 | Toluene | S < 0.9 |
| EtOAc | 1.1 < S < 1.2 | n-heptane | S < 0.9 |
| IPAc | S < 1.0 | DMAc | S > 36.0 |
| MTBE | S < 1.0 | H₂O | S < 1.0 |
| THF | 9.5 < S < 19.0 | CHCl₃ | 10.5 < S < 21.0 |

TABLE 10
Summary of polymorph screening for Compound 2 Type II

| | No. of | |
|---|---|---|
| Method | experiment | Crystal form |
| Anti-solvent addition | 21 | Compound 1 Type A |
| | | Compound 1 Type B |
| | | Compound 2 Type I |
| | | Compound 2 Type II |
| Solid vapor diffusion | 13 | Compound 2 Type II |
| Solution vapor diffusion | 12 | Compound 1 Type A |
| Polymer-induced crystallization | 10 | Compound 2 Type I |
| Slow evaporation | 10 | Compound 2 Type II |
| Slurry at room temperature | 12 | Compound 1 Type B |
| | | Compound 2 Type II |
| Slurry at 5° C. | 12 | Compound 1 Type A |
| | | Compound 1 Type B |
| | | Compound 2 Type II |
| Slow cooling | 8 | Compound 1 Type A |
| | | Compound 2 Type II |
| Grinding | 2 | Compound 2 Type II |
| Total | 100 | Compound 1 Type A |
| | | Compound 1 Type B |
| | | Compound 2 Type I |
| | | Compound 2 Type II |

### 1. Anti-Solvent Addition

A total of 21 anti-solvent addition experiments were carried out. About 15 mg of Compound 2 Type II was dissolved in 0.1-2.4 mL solvent to obtain a clear solution. The solution was magnetically stirred, then followed by addition of 0.1 mL anti-solvent per step until a precipitate appeared or the total amount of anti-solvent reached 15.0 mL. The precipitate was isolated for XRPD analysis. Clear solutions were transferred to agitation at 5° C. for 1 day, and solids were then tested by XRPD. The final clear solutions were transferred to evaporation at room temperature. Results are summarized in Table 11.

TABLE 11
Summary of anti-solvent addition experiments

| Solvent | Anti-solvent | Solid Form |
|---|---|---|
| MeOH | H₂O | Compound 1 Type B |
| THF | | Compound 1 Type B |
| EtOH* | n-heptane | Compound 1 Type A |
| IPA* | | Compound 1 Type A |
| Acetone | | Compound 1 Type A |
| | | Compound 2 Type II |
| THF | | Compound 1 Type A |
| | | Compound 2 Type II |
| 2-MeTHF | | Compound 1 Type B + |
| | | Compound 2 Type II |
| 1,4-Dioxane | | Compound 1 Type A |
| | | Compound 2 Type II |
| DCM | | Compound 2 Type II |
| CHCl₃ | | Compound 1 Type A |
| | | Compound 2 Type I |
| MeOH** | Toluene | Compound 1 Type A |
| | | Compound 2 Type I |
| THF* | | Compound 2 Type II |
| CHCl₃ | | Compound 1 Type A |
| | | Compound 2 Type I |
| | | Compound 2 Type II |
| Acetone* | | Compound 2 Type II |
| DMSO* | | Compound 1 Type B |
| NMP** | IPAc | Compound 1 Type A |
| | | Compound 2 Type II |
| THF** | | Compound 2 Type II |
| EtOH** | | Compound 1 Type A |
| | | Compound 2 Type II |
| MeOH* | MTBE | Compound 2 Type II |
| DMAc* | | Compound 1 Type A |
| CHCl₃ | | Compound 1 Type A |

*Solids were obtained via cooling at 5° C.
**Solids were obtained via evaporation.

### 2. Solid Vapor Diffusion

Solid vapor diffusion experiments were conducted using 13 different kinds of solvent. Approximately 10 mg of Compound 2 Type II was weighed into a 3-mL vial, which was placed into a 20-mL vial with 2 mL of relative solvent. The 20-mL vial was sealed with a cap and kept at room temperature allowing solvent vapor to interact with sample for 7 days. The solids were tested by XRPD and the results were summarized in Table 12 which showed that only Compound 2 Type II was observed.

TABLE 12
Summary of solid vapor diffusion experiments

| Solvent | Solid Form |
|---|---|
| H₂O | Compound 2 Type II |
| DCM | Compound 2 Type II |
| EtOH | Compound 2 Type II |
| MeOH | Compound 2 Type II |
| ACN | Compound 2 Type II |
| THF | Compound 2 Type II |
| CHCl₃ | Compound 2 Type II |
| Acetone | Compound 2 Type II |
| DMF | Compound 2 Type II |
| EtOAc | Compound 2 Type II |
| 1,4-Dioxane | Compound 2 Type II |
| IPA | Compound 2 Type II |
| DMSO | Compound 2 Type II |

### 3. Solution Vapor Diffusion

12 solution vapor diffusion experiments were conducted. Approximate 15 mg of Compound 2 Type II was dissolved in 0.6-2.4 mL of appropriate solvent to obtain a clear solution in a 3-mL vial. This solution was then placed into a 20-mL vial with 3 mL of relative solvents. The 20-mL vial was sealed with a cap and kept at room temperature allowing sufficient time for organic vapor to interact with the solution. The precipitates were isolated for XRPD analysis. The results summarized in Table 13 showed that Compound 1 Type A and Compound 2 Type I and Type II were observed.

TABLE 13
Summary of solution vapor diffusion experiments

| Solvent | Anti-solvent | Solid Form |
|---|---|---|
| MeOH | toluene | Compound 2 Type II |
| THF | | Compound 1 Type A |
| | | Compound 2 Type I |
| | | Compound 2 Type II |
| Acetone | | Compound 1 Type A |
| | | Compound 2 Type II |
| DCM | | Compound 1 Type A |
| | | Compound 2 Type II |
| Acetone | n-heptane | Compound 1 Type A |
| | | Compound 2 Type II |
| 2-MeTHF | | Compound 1 Type A |
| EtOH | | Compound 1 Type A |
| | | Compound 2 Type I |
| | | Compound 2 Type II |
| DCM | | Compound 1 Type A |
| | | Compound 2 Type II |
| CHCl₃ | | Compound 2 Type I |
| | | Compound 2 Type II |
| EtOH | IPAc | Compound 1 Type A |
| | | Compound 2 Type II |
| THF | | Compound 1 Type A |
| | | Compound 2 Type II |
| CHCl₃ | MTBE | Compound 2 Type II |

### 4. Polymer-Induced Crystallization

Polymer-induced crystallization experiments were performed with two sets of polymer mixtures in five different solvents. Approximately 15 mg of Compound 2 Type II sample was dissolved in 0.8-2.0 mL of appropriate solvent to obtain a clear solution in a 3-mL vial. About 2 mg of polymer mixture was added into 3-mL glass vial. All the samples were sealed using parafilm and then transferred to evaporation at room temperature to induce precipitation. The solids were isolated for XRPD analysis. Results summarized in Table 14 showed that Compound 1 Type A, and Compound 2 Type I and II were produced.

TABLE 14
Summary of polymer-induced crystallization experiments

| Solvent (v/v) | Polymer | Solid Form |
|---|---|---|
| EtOH | Mixture A | Compound 1 Type A |
| | | Compound 2 Type II |
| Acetone | | Compound 1 Type A |
| | | Compound 2 Type II |
| THF | | Compound 1 Type A |
| | | Compound 2 Type II |
| DCM | | Compound 1 Type A |
| | | Compound 2 Type II |
| MeOH/toluene (4:1) | | Compound 2 Type II |
| EtOH | Mixture B | Compound 1 Type A |
| | | Compound 2 Type I |
| | | Compound 2 Type II |
| Acetone | | Compound 2 Type II |
| THF | | Compound 1 Type A |
| | | Compound 2 Type I |
| | | Compound 2 Type II |
| DCM | | Compound 1 Type A |
| | | Compound 2 Type I |
| | | Compound 2 Type II |
| MeOH/toluene (4:1) | | Compound 2 Type II |

Polymer mixture A: polyvinyl pyrrolidone (PVP), polyvinyl alcohol (PVA), polyvinylchloride (PVC), polyvinyl acetate (PVAC), hypromellose (HPMC), methyl cellulose (MC) (mass ratio of 1:1:1:1:1:1)
Polymer mixture B: polycaprolactone (PCL), polyethylene glycol (PEG), poly(methyl methacrylate) (PMMA) sodium alginate (SA), and hydroxyethyl cellulose (HEC) (mass ratio of 1:1:1:1:1).

### 5. Slow Evaporation

Evaporation experiments were performed under 10 conditions. Briefly, ˜15 mg of Compound 2 Type II were dissolved in 0.7-2.5 mL of corresponding solvent in a 3-mL glass vial. The visually clear solutions were subjected to evaporation at room temperature to induce precipitation. The solids were isolated for XRPD analysis, and the results summarized in Table 15 indicated that that Compound 1 Type A, and Compound 2 Types I and II were produced.

TABLE 15
Summary of evaporation experiments

| Solvent (v/v) | Solid Form |
|---|---|
| MeOH | Compound 2 Type II |
| EtOH | Compound 1 Type A |
| IPA | Compound 1 Type A |
| | Compound 2 Type II |
| Acetone | Compound 1 Type A |
| | Compound 2 Type I |
| | Compound 2 Type II |
| THF | Compound 1 Type A |
| | Compound 2 Type II |
| 2-MeTHF | Compound 1 Type A |
| 1,4-dioxane | Compound 1 Type A |
| | Compound 2 Type II |
| DCM | Compound 1 Type B |
| | Compound 2 Type II |
| CHCl₃/toluene (4:1) | Compound 1 Type A |
| | Compound 2 Type I |
| | Compound 2 Type II |
| THF/n-heptane (4:1) | Compound 1 Type A |

### 6. Slurry at Room Temperature

Slurry conversion experiments were conducted at room temperature in 12 different solvent systems. About 15 mg of Compound 2 Type II was suspended in 0.5 mL of solvent in a 1.5-mL glass vial. After the suspension was stirred at room temperature for about 4 days, the remaining solids were isolated for XRPD analysis. Results summarized in Table 16 indicated that Compound 1 Type B and Compound 2 Type II were generated.

TABLE 16
Summary of slurry conversion experiments at room temperature

| Solvent (v/v) | Solid Form |
|---|---|
| H₂O | Compound 1 Type B |
| | Compound 2 Type II |
| IPA | Compound 2 Type II |
| Acetone | Compound 2 Type II |
| 2-MeTHF | Compound 2 Type II |
| DCM | Compound 2 Type II |
| MTBE | Compound 2 Type II |
| EtOAc | Compound 2 Type II |
| ACN | Compound 2 Type II |
| THF/n-heptane(1:5) | Compound 2 Type II |
| CHCl₃/toluene(1:5) | Compound 2 Type II |
| MeOH/toluene(1:9) | Compound 2 Type II |
| EtOH/n-heptane(1:5) | Compound 2 Type II |

### 7. Slurry at 50° C.

Slurry conversion experiments were also conducted at 50° C. in 12 different solvent systems. About 15 mg of Compound 2 Type II was suspended in 0.5 mL of solvent in a 1.5-mL glass vial. After the suspension was stirred at 50° C. for about 4 days, the remaining solids were isolated for XRPD analysis. Results summarized in Table 17 indicate that Compound 1 Type A and B, and Compound 2 Type II were generated.

TABLE 17
Summary of slurry conversion experiments at 50° C.

| Solvent (v/v) | Solid Form |
|---|---|
| H₂O | Compound 1 Type B |
| | Compound 2 Type II |
| MIBK | Compound 2 Type II |
| EtOAc | Compound 2 Type II |
| ACN | Compound 2 Type II |
| n-Octanol | Compound 2 Type II |
| Cyclohexane | Compound 2 Type II |
| MTBE | Compound 2 Type II |
| Acetone/n-heptane(1:5) | Compound 2 Type II |
| THF/n-heptane(1:5) | Compound 2 Type II |
| CHCl₃/toluene(1:5) | Compound 2 Type II |
| MeOH/toluene(1:9) | Compound 2 Type II |
| EtOH/n-heptane(1:5) | Compound 2 Type II |

### 8. Slow Cooling

Slow cooling experiments were conducted in eight solvent systems. About 20 mg of Compound 2 Type II was suspended in 1.0 mL of solvent in a 3-mL glass vial at room temperature. The suspension was then heated to 50° C., equilibrated for 2 hrs, and filtered to a new vial using a Nylon membrane (pore size of 0.45 μm). Filtrates were slowly cooled down to 5° C. at a rate of 0.1° C./min. The obtained solids were kept isothermal at 5° C. before isolated for XRPD analysis. Clear solutions were evaporated to dryness at 5° C. and then solids were tested by XRPD. Results summarized in Table 18 indicated Compound 1 Type A and Compound 2 Type II were obtained.

TABLE 18
Summary of slow cooling experiments

| Solvent (v/v) | Solid Form |
|---|---|
| IPA* | Compound 2 Type II |
| Acetone | Compound 1 Type A |
| | Compound 2 Type II |
| 2-MeTHF | Compound 1 Type A |
| | Compound 2 Type II |
| IPAc* | Compound 1 Type A |
| THF/n-heptane (1:1) | Compound 1 Type A |
| | Compound 2 Type II |
| THF/toluene (1:5)* | Compound 1 Type A |
| | Compound 2 Type II |
| CHCl₃/n-heptane (1:3) | N/A |
| CHCl₃/toluene (1:3)* | Compound 1 Type A |

N/A: no solid was obtained.
*Solids were obtained via evaporation.

### 9. Grinding

Grinding induced phase transition experiments was performed in two conditions with/without additive. About 15 mg of Compound 2 Type II was weighed into a mortar and then ground manually using a pestle for 5 mins. The solid was analyzed by XRPD and no new crystal form was generated (Table 19).

TABLE 19
Summary of grinding experiments

| Additive | Solid Form |
|---|---|
| N/A | Compound 2 Type II |
| H₂O | Compound 2 Type II |

N/A: no additive was added.

### Example 10

### Protocol for Human B Cell Stimulation

Human B cells are purified from 150 ml of blood. Briefly, the blood can be diluted 1/2 with PBS and centrifuged through a Ficoll density gradient. The B cells can be isolated from the mononuclear cells by negative selection using the B cell isolation kit II from Milenyi (Auburn, Calif.). 50,000 B cells per well can then be stimulated with 10 μg/ml of goat F(ab′)2 anti-human IgM antibodies (Jackson ImmunoResearch Laboratories, West Grove, Pa.) in a 96-well plate. Compounds can be diluted in DMSO and added to the cells. Final concentration of DMSO is 0.5%. Proliferation can be measured after 3 days using Promega CellTiter-Glo (Madison, Wis.).

### Example 11

### In Vitro BTK Kinase Assay: BTK-POLYGAT-LS ASSAY

The purpose of the BTK in vitro assay is to determine compound potency against BTK through the measurement of IC₅₀. Compound inhibition can be measured after monitoring the amount of phosphorylation of a fluorescein-labeled polyGAT peptide (Invitrogen PV3611) in the presence of active BTK enzyme (Upstate 14-552), ATP, and inhibitor. The BTK kinase reaction can be done in a black 96 well plate (costar 3694). For a typical assay, a 24 μL aliquot of a ATP/peptide master mix (final concentration; ATP 10 μM, polyGAT 100 nM) in kinase buffer (10 mM Tris-HCl pH 7.5, 10 mM MgCl₂, 200 μM Na₃PO₄, 5 mM DTT, 0.01% Triton X-100, and 0.2 mg/ml casein) can be added to each well. Next, 1 μL of a 4-fold, 40× compound titration in 100% DMSO solvent can be added, followed by addition of 15 uL of BTK enzyme mix in 1× kinase buffer (with a final concentration of 0.25 nM). The assay can be incubated for 30 minutes before being stopped with 28 μL of a 50 mM EDTA solution. Aliquots (5 μL) of the kinase reaction can be transferred to a low volume white 384 well plate (Corning 3674), and 5 μL of a 2× detection buffer (Invitrogen PV3574, with 4 nM Tb-PY20 antibody, Invitrogen PV3552) can be added. The plate can be covered and incubated for 45 minutes at room temperature. Time resolved fluorescence (TRF) on Molecular Devices M5 (332 nm excitation; 488 nm emission; 518 nm fluorescein emission) can be measured. IC₅₀ values can be calculated using a four parameter fit with 100% enzyme activity determined from the DMSO control and 0% activity from the EDTA control.

### Example 12

### In Vitro Inhibition of BTK Activity in Mouse Whole Blood

Anti-rabbit MSD plates (Meso Scale Discovery, Rockville, Md.) can be coated with 35 μL/well of rabbit anti-BTK C82B8 (Cell Signaling Technology, Danvers, Mass.) diluted 1:50 in PBS. Plates can be incubated for 2 hours ±1 hour at room temp, shaking (setting 3-5) or ON at 4° C. Plates can be blocked with MSD Blocker A (Meso Scale Discovery, Rockville, Md.) using 3% MSD Blocker A in TBST. Coated plates can be first washed 3× with 250 uL/well TBST followed by addition of 200 uL/well 3% Blocker A/TBST. Plates can be blocked for >2 hour at room temperature, shaking or ON at 4° C.

Whole blood can be collected from DBA/1 mice in 16×100 sodium heparin tubes (Becton Dickinson, Cat No. 367874). Blood from multiple DBA/1 mice can be pooled. 96 uL of whole blood per well can be aliquotted into a 96-round bottom plate changing tips each time. 4 uL diluted test compound can be added to each sample, mixed, and incubated for 30 min at 37° C.

For serial dilutions of test compound, 1000× plate can be produced with serial dilutions of test compound in 100% DMSO. Ten dilutions, done 1:3, starting at 10 mM can be created by: adding 15 uL of test compound at 10 mM in 100% DMSO to well A1; adding 10 uL 100% DMSO to wells A2-A12; diluting 5 uL from well A1 to well A2 and mixing; continuing 1:3 serial dilutions, changing tips between transfers, to well A10. Wells A11 and A12 can contain 100% DMSO without test compound.

For dilution 1, a 1:40 plate can be created. Using a 12-well multi-channel pipette, each concentration of test compound or DMSO can be diluted 1:40 by adding 2 uL from each well of 1000× stock plate to 78 uL water and mixing.

For dilution 2, test compound or DMSO can be added to whole blood by diluting 1:25. Using a 12-well multi-channel pipette, 4 uL from 1:40 plate (B) can be added to 96 uL whole blood and mixed.

Lysing buffer used to lyse whole blood can be prepared as follows. A 10× Lysis buffer can be prepared using 1500 mM NaCl; 200 mM Tris, pH 7.5; 10 mM EDTA; 10 mM EGTA; and 10% Triton-X-100. The 10× Lysis buffer is diluted to 1× in dH₂O, and complete lysing buffer (+/− phosphatase inhibitors) can be prepared as follows:

| | +PPi (mL) | −PPi (mL) |
|---|---|---|
| 1X Lysis buffer | 10 | 10 |
| 500 mM PMSF in DMSO | 0.02 | 0.02 |
| Phosphatase Inhibitor 3 | 0.1 | |
| Phosphatase Inhibitor 2 | 0.1 | |
| Protease Inhibitor (cOmplete) (1 tablet for 10 mL) | 1 tablet | 1 tablet |
| PhosStop (1 tablet for 10 mL) | 1 tablet | |
| Sodium Orthovanadate (Na₃VO₄) (50 uM final) | 0.1 | |
| Sodium Fluoride (NaF) (10 mM final) | 0.005 | |
| 1% Deoxycholate (0.25% final) | 2.5 | 2.5 |

100 uL of complete lysing buffer (+/− phosphatase inhibitors) can be added to each well, and mixed well by pipetting up and down a few times. Wells 1-10 and 12 can receive 1× Lysis buffer containing phosphatase inhibitors (+PPi) and well 11 can receive 1× Lysis buffer without phosphatase inhibitors (−PPi). Samples can be incubated for 1 hour on ice or at 4° C. Samples can be mixed again at half time point for complete lysing.

Blocking buffer can be washed off blocked MSD plates with 250 uL TBST per well 3 times. 100-150 uL of whole blood lysates can be added to each well of the coated and blocked MSD plates followed by incubation overnight in a cold room with shaking.

The plates can then washed 4 times with 250 μL TBST per well. Biotinylated phospho-tyrosine mouse mAb (pY100, Cell Signaling Technology, Danvers, Mass.) can be diluted 1:125 in 1% Blocker A. Mouse anti-BTK mAb (Fitzgerald Industries International, Acton, Mass.) can be diluted 1:900 in 1% Blocker A. 35 μL of diluted pY100 or diluted anti-BTK mAb can be added to each well and incubated for 2 hours at room temperature, shaking.

Plates can be then washed 3 times with 250 uL TBST/well. 35 uL of 1:500 Streptavidin-Sulfo-Tag labeled antibody in 3% Blocker A can be added to each well. For anti-BTK, 35 uL of 1:500 anti-mouse-Tag labeled antibody in 3% Blocker A can be added to each well. Plates can be incubated for 1 hour at room temperature, shaking.

To develop and read the plates, 1× Read Buffer in dH₂O can be prepared from 4× stock. Plates can be washed 3 times with 250 uL TBST/well. 150 uL of 1× MSD Read Buffer is added to each well. Plates can be read in a SECTOR Imager 6000 (Meso Scale Discovery, Rockville, Md.).

### Materials

| ITEM | VENDOR | CATALOG NO. |
|---|---|---|
| Anti-rabbit MSD plates | MSD | L45RA-1 |
| Rabbit anti-BTK (C82B8) | Cell Signaling | 3533S |
| PBS | Media Prep | |
| MSD Blocker A | MSD | R93BA-4 |
| TBST (1xTBS/0.1% Tween20) | Media Prep | |
| 10X Lysing Buffer | Media Prep | |
| PMSF in DMSO (500 mM) | Media Prep | |
| Phosphatase Cocktail Inhibitor 3 | Sigma Aldrich | P0044-5ML |
| Phosphatase Cocktail Inhibitor 2 | Sigma Aldrich | P5726-1ML |
| cOmplete Mini | Roche | 11 836 153 001 |
| PhosStop Inhibitor | Roche | 04 906 837 001 |
| Sodium Orthovanadate 100 mM | Media Prep | |
| Sodium Fluoride 1M | Media Prep | |
| 1% Deoxycholate | Media Prep | |
| pTyr 100 ms mAb biotinylated | Cell Signaling | 9417S |
| Streptavidin Sulfo-Tag | MSD | R32AD-1 |
| MSD Read Buffer 4X | MSD | R92TC-1 |
| Costar 96-round bottom | Costar/Fisher | 3799 |
| Mouse anti-BTK (7F12H4) | Fitzgerald | 10R-1929 |
| Anti-mouse Sulfo-Tag | MSD | R32AC-5 |

### Example 13

### PK/PD Correlation in DBA1 Mice

Mice can be dosed orally (PO) with test compound in CMC-Tween and killed by CO₂ asphyxiation at various times after dosing. Heparinized whole blood can be immediately collected by cardiac puncture and split into two samples. One sample can be used to quantify the amount of test compound present and the other is lysed in MSD lysis buffer in the presence of phosphatase inhibitors. Heparinized whole blood from cardiac punctures of vehicle (CMC-Tween) dosed mice can be lysed either in the presence (high control) or absence (low control) of phosphatase inhibitors. Lysed whole blood samples can be analyzed for phospho-BTK as described above. The percent inhibition of phospho-BTK in each whole blood sample from dosed mice can be calculated as follows: (1−((pBTK(x+PPi)−pBTK(vehicle−PPi))/(pBTK(vehicle+PPi))))*100, where pBTK(x+PPi) is the ECL signal for whole blood from each test compound-treated mouse, pBTK(vehicle −PPi) is the average ECL signal of whole blood from vehicle-treated mice lysed in the absence of phosphatase inhibitors (low control) and pBTK(vehicle+PPi) is the average ECL signal of whole blood from vehicle-treated mice lysed in the presence of phosphatase inhibitors (high control).

### Example 14

### In Vitro PD Assay in Human Whole Blood

Human heparinized venous blood can be purchased from Bioreclamation, Inc. or SeraCare Life Sciences and shipped overnight. Whole blood can be aliquoted into 96-well plate and “spiked” with serial dilutions of test compound in DMSO or with DMSO without drug. The final concentration of DMSO in all wells can be 0.1%. The plate can be incubated at 37° C. for 30 min. Lysis buffer containing protease and phosphatase inhibitors can be added to the drug-containing samples and one of the DMSO-only samples (+PPi, high control), while lysis buffer containing protease inhibitors can be added to the other DMSO-only samples (−PPi, low control). All of the lysed whole blood samples can be subjected to the total BTK capture and phosphotyrosine detection method described in Example 12. ECL values can be graphed in Prism and a best-fit curve with restrictions on the maximum and minimum defined by the +PPi high and −PPi low controls can be used to estimate the test compound concentration that results in 50% inhibition of ECL signal by interpolation.

### Example 15

### Preparation of Single Crystals of Compound 2 Type I

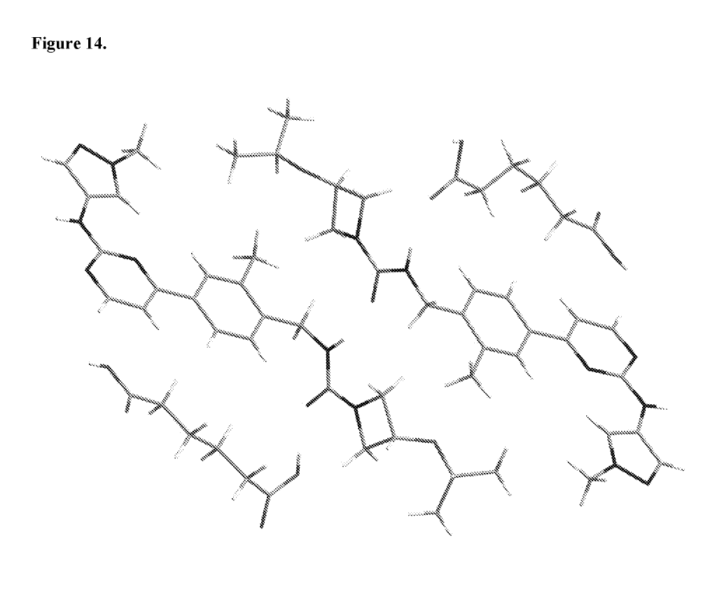

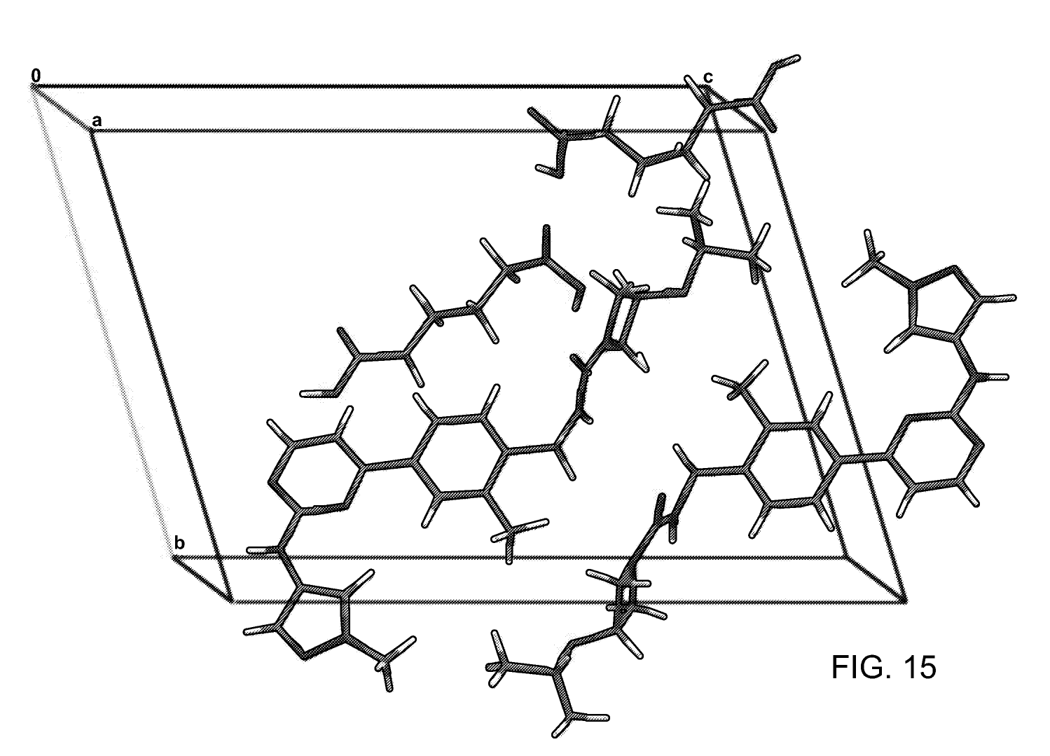

10.2 mg Compound 2 Type II and 3.6 mg adipic acid were weighed into a 3 mL vial with addition of 0.6 mL dichloromethane/acetonitrile (1/5, v/v) mixture solvent. The solution was vortexed and sonicated for 3 minutes. The vial was kept in a 50° C. oven and heated for 0.5 h, then the solution was filtered with a 0.45 μm filter to another two 3 mL vials that were preheated at 50° C. Seeds of Compound 2 Type I were added in the vials that contained the filtrate. The vials were then placed in a 50° C. bio-chemical incubator and cooled down from 50° C. to 5° C. at the speed of 0.01° C./min. After five days, rod-like crystals were obtained. The three-dimensional structure of Compound 2 Type I single crystal and the unit cell of Compound 2 Type I single crystal are shown in FIG. 14 and FIG. 15, respectively. The details of the crystal data and structure refinement are listed in Table 20.

TABLE 20
Summary of crystal data and structure refinement.

| | |
|---|---|
| Empirical formula | C₂₉H₃₉N₇O₆ |
| Formula weight | 581.67 |
| Temperature | 298(2) K |
| Wavelength | 0.71073 Å |
| Crystal system, space group | Triclinic, P 1 |
| Unit cell dimensions | a = 5.2557(8) Å |
| | b = 14.935(3) Å |
| | c = 20.489(3) Å |
| | α = 70.370(6)° |
| | β = 85.120(4)° |
| | γ = 84.901(4)° |
| Volume | 1506.2(4) Å³ |
| Z, Calculated density | 2, 1.283 Mg/m³ |
| Absorption coefficient | 0.092 mm⁻¹ |
| F(000) | 620 |
| Crystal size | 0.30 × 0.26 × 0.24 mm |
| Theta range for data collection | 2.13 to 25.00° |
| Limiting indices | −6 ≤ h ≤ 6 |
| | −17 ≤ k ≤ 17 |
| | −24 ≤ l ≤ 24 |
| Reflections collected/unique | 47593/5261 [R(int) = 0.0766] |
| Completeness | 98.9% |
| Refinement method | Full-matrix least-squares on F² |
| Data/restraints/parameters | 5261/24/759 |
| Goodness-of-fit on F² | 1.057 |
| Final R indices [I > 2sigma(I)] | R₁ = 0.0581, wR₂ = 0.1439 |
| Largest diff. peak and hole | 0.271 and −0.226 e.A⁻³ |

### Example 16

### Preparation of Single Crystals of Compound 2 Type II

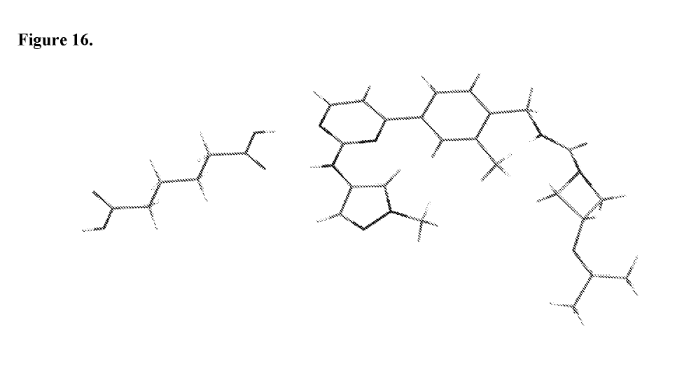

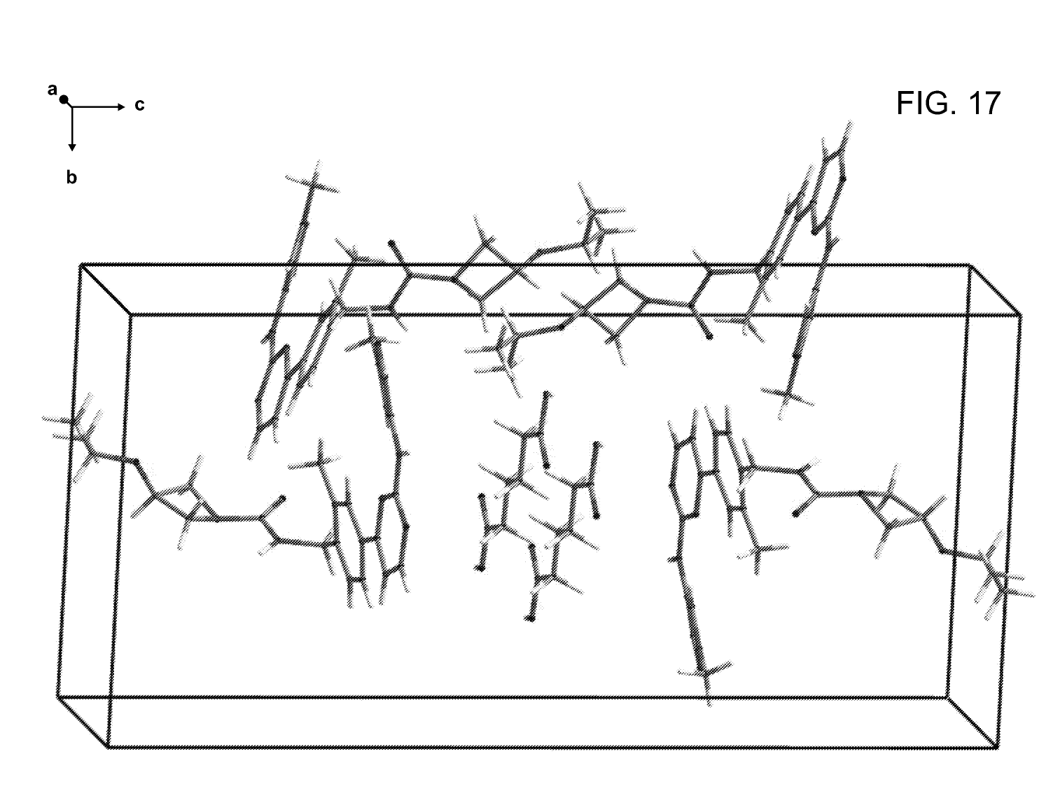

5.0 mg Compound 2 Type II was weighed into a 3 mL vial with addition of 0.8 mL methyl isobutyl ketone. The solution was vortexed and sonicated for 3 minutes. The vial was then kept in a 50° C. oven and heated for 0.5 h, then the solution was filtered with a 0.45 μm filter to another 3 mL vial that have be preheated at 50° C., seeds of Compound 2 Type II were added in the vial that contained the filtrate, to induce any crystal growth. The vial was then moved into a 50° C. bio-chemical incubator and cooled down from 50° C. to 5° C. at the speed of 0.01° C./min. After three days, rod-like crystals were obtained. The three-dimensional structure of Compound 2 Type II single crystal and the unit cell of Compound 2 Type II single crystal are shown in FIG. 16 and FIG. 17, respectively. The details of the crystal data and structure refinement are listed in Table 21.

TABLE 21
Summary of crystal data and structure refinement.

| | |
|---|---|
| Empirical formula | C₂₆H₃₄N₇O₄ |
| Formula weight | 508.60 |
| Temperature | 123(2) K |
| Wavelength | 0.71073 Å |
| Crystal system, space group | Monoclinic, P2₁/n |
| Unit cell dimensions | a = 6.7183(6) Å |
| | b = 13.8744(11) Å |
| | c = 28.383(2) Å |
| | α = 90° |
| | β = 91.220(3)° |
| | γ = 90° |
| Volume | 2645.0(4) Å³ |
| Z, Calculated density | 4, 1.277 Mg/m³ |
| Absorption coefficient | 0.089 mm⁻¹ |
| F(000) | 1084 |
| Crystal size | 0.32 × 0.24 × 0.23 mm |
| Theta range for data collection | 2.94 to 25.00° |
| Limiting indices | −7 ≤ h ≤ 7 |
| | −16 ≤ k ≤ 16 |
| | −33 ≤ l ≤ 33 |
| Reflections collected/unique | 60197/4634 [R(int) = 0.0462] |
| Completeness | 99.8% |
| Refinement method | Full-matrix least-squares on F² |
| Data/restraints/parameters | 4634/0/336 |
| Goodness-of-fit on F² | 0.995 |
| Final R indices [I > 2sigma(I)] | R₁ = 0.0797, wR₂ = 0.2189 |
| Largest diff. peak and hole | 1.749 and −0.523 e.A⁻³ |

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A solid form Compound 2 comprising Compound 1 and adipic acid:

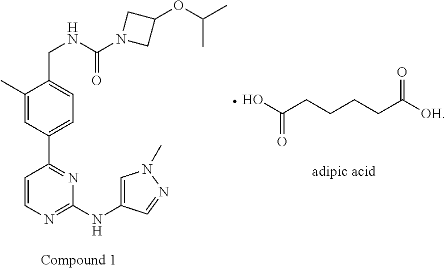 [SMILES: Cc1cc(-c2ccnc(Nc3cnn(C)c3)n2)ccc1CNC(=O)N1CC(OC(C)C)C1 ; O=C(O)CCCCC(=O)O]

2. The solid form according to claim 1, wherein said solid form is a crystalline solid that is a co-crystal of Compound 1 and adipic acid.

3. The solid form according to claim 2, wherein said crystalline solid is substantially free of amorphous Compound 2.

4. The solid form according to claim 1, wherein said solid form is substantially free of impurities.

5. The solid form according to claim 2, wherein said solid form is of Type I.

6. The solid form according to claim 5, having one or more peaks in its XRPD selected from those at about 6.34, about 9.24, and about 27.37 degrees 2-theta.

7. The solid form according to claim 6, having at least two peaks in its XRPD selected from those at about 6.34, about 9.24, and about 27.37 degrees 2-theta.

8. The solid form according to claim 5, having a XRPD substantially similar to that depicted in FIG. 5.

9. The solid form according to claim 2, wherein said compound is of Type II.

10. The solid form according to claim 9, having one or more peaks in its XRPD selected from those at about 7.04, about 20.08, and about 25.14 degrees 2-theta.

11. The solid form according to claim 10, having at least two peaks in its XRPD selected from those at about 7.04, about 20.08, and about 25.14 degrees 2-theta.

12. The solid form according to claim 9, having a XRPD substantially similar to that depicted in FIG. 10.

13. The solid form of claim 1, wherein said solid form is an amorphous solid form.

14. The solid form according to claim 13, wherein said solid form is substantially free of crystalline Compound 2.

15. The compound according to claim 13, wherein said solid form is substantially free of impurities.

16. A composition comprising the solid form according to any one of claims 1-15 and a pharmaceutically acceptable carrier or excipient.

17. A method of decreasing the enzymatic activity of Bruton's tyrosine kinase comprising contacting Bruton's tyrosine kinase with an effective amount of the solid form of any one of claims 1-15 or a composition thereof.

18. A method of treating a disorder responsive to inhibition of Bruton's tyrosine kinase comprising administering to a subject an effective amount of the solid form of any one of claims 1-15 or a composition thereof.

19. A method of treating a disorder selected from the group consisting of autoimmune disorders, inflammatory disorders, and cancers comprising administering to a subject an effective amount of the solid form of any one of claims 1-15 of a composition thereof.

20. The method according to claim 19, wherein the disorder is selected from rheumatoid arthritis, systemic lupus erythematosus, atopic dermatitis, leukemia and lymphoma.
